(19) United States
(12) Patent Application Publication
(10) Pub. No.: US 2026/0038668 A1
(43) Pub. Date: Feb. 5, 2026

(54) DYNAMIC ACTIVITY RECOMMENDATION USING MACHINE LEARNING AND GEOFENCING IN A MENTAL WELLNESS APPLICATION

(71) Applicant: Brightn, Inc., Cedar Rapids, IA (US)

(72) Inventors: Jeffrey Andrew Johnston, Cedar Rapids, IA (US); Carson Goodale, Cedar Rapids, IA (US)

(21) Appl. No.: 18/792,249

(22) Filed: Aug. 1, 2024

(51) Int. Cl. G16H 20/70; G16H 10/20; G16H 10/60

(52) U.S. Cl. CPC G16H 20/70; G16H 10/20; G16H 10/60

(57) ABSTRACT

Techniques described herein include receiving a user request to create a personal wellness plan. The user may then be provided with questions that have been determined to be associated with a health asset class, wealth asset class, and/or purpose asset class. Based on receiving user input data representing a response to the questions, the user input data may be used to determine appropriate activities to recommend to the user, where the activities are also associated with the health asset class, wealth asset class, and/or purpose asset class. User input data and/or user activity data may then be utilized to train a model configured for determining subsequent questions and activities to present to the user. User location data may also be used to determine appropriate activities based on an activity being within a user's geofence.

### TECHNICAL FIELD

Applications, which are downloadable and executable on user devices, enable users to engage in guided activities, such as exercise and/or meditation. This disclosure relates generally to dynamic activity recommendation using machine learning and geofencing in a mental wellness application.

### BRIEF DESCRIPTION OF THE DRAWINGS

Features of the present disclosure, its nature and various advantages, will be more apparent upon consideration of the following detailed description, taken in conjunction with the accompanying drawings. The detailed description is described with reference to the accompanying figures. In the figures, the left-most digit(s) of a reference number identify the figure in which the reference number first appears. The same reference numbers in different figures indicate similar or identical items. The systems depicted in the accompanying figures are not to scale and components within the figures may be depicted not to scale with each other.

FIG. 1 illustrates a schematic diagram of an illustrative environment for dynamically determining activity recommendation outputs responsive to user input of a mobile device user, according to at least some examples of the present disclosure.

FIG. 2 illustrates example components of the remote system of FIG. 1 that performs an example of processing user input data to determine an activity that corresponds to the user input data, and dynamically updating activity based at least in part on further user input, machine learning processing, and/or geofencing, according to at least some examples of the present disclosure.

FIG. 3 illustrates an example user interface for the determination of dynamic activity recommendations for responding to user input, according to at least some examples of the present disclosure.

FIGS. 4A and 4B illustrate a conceptual diagram associated with the use of geofencing to dynamically select activity recommendations for responding to user input, according to at least some examples of the present disclosure.

FIG. 5 illustrates a flow diagram of an example process for determining activity recommendation outputs in response to user input received in association with a user input device, according to at least some examples of the present disclosure.

FIG. 6 illustrates a flow diagram of another example process for determining activity recommendation outputs in response to user input received in association with a user input device, according to at least some examples of the present disclosure.

FIG. 7 illustrates a flow diagram of an example process for generation of a machine learning model and the use of the same.

FIG. 8 illustrates an example user interface of the personalized weekly plans in response to user inputs, according to at least some examples of the present disclosure.

FIG. 9 illustrates an example user interface for the selection of challenges and opportunities integrated into weekly mental wellness plans, according to at least some examples of the present disclosure.

FIG. 10 illustrates an example user interface of multiple daily essential modules, according to at least some examples of the present disclosure.

FIG. 11 illustrates an example user interface of a mood tracker for collecting user input responses, according to at least some examples of the present disclosure.

FIG. 12 illustrates an example user interface of an index for tracking mental wellness improvements in response to user inputs, according to at least some examples of the present disclosure.

FIG. 13 illustrates an example user interface of the application for the creation of unique life statements in response to user inputs, according to at least some examples of the present disclosure.

FIG. 14 illustrates an example user interface of generated push notifications in response to user inputs in the communication system, according to at least some examples of the present disclosure.

FIG. 15 illustrates an example user interface of the social platform within the mental wellness application, according to at least some examples of the present disclosure.

### DETAILED DESCRIPTION

Described herein are, at least in part, techniques including processing user input data using machine learning and/or geofencing to dynamically change user content and provide an appropriate activity recommendation output from among multiple activities associated with health, wellness, and/or purpose asset classes. The techniques described herein may be applicable in various scenarios, including scenarios where a user would like to create a dynamic personal wellness plan, and where an updated personal wellness plan is generated daily or otherwise periodically, and/or in real time or near real time, to reflect user input data and/or user activity data. A remote system may store a library of questions, where each question may be associated with a specific asset class such as health, wealth, and/or purpose. Further, the remote system may store a library of activities, where each activity may be associated with a specific asset class such as health, wealth, and/or purpose. For example, activities associated with the health asset class may include running or biking. Activities associated with the wealth asset class may include opening a savings account or starting a budget plan, for example. Activities associated with the purpose asset class may include learning about breath work or watching a video on meditation, for example.

In an example, and not by way of limitation, a user may interact with a user device, such as a smartphone, and using an application operating on the user device. In such example, the user may submit a request to create the personal wellness plan as part of making a user account in association with the application. The user may then be presented with first questions retrieved from the library of questions, where there is at least one question associated with each of the health, wealth, and/or purpose asset classes. For example, a user may be presented with nine questions: three questions from the health asset class; three questions from the wealth asset class; and three questions from the purpose asset class. The user may then provide user input (e.g., responses) to each of the questions.

In an example, user input data representing the user input may be received by the remote system, where each question in the library of questions may be associated with one or more activities from the library of activities. Based on the user input data, the user may then be provided with recommended activities that are responsive to the user input and associated with the health, wealth, and/or purpose asset classes. For example, the user may be provided with a health asset class activity based at least in part on the user's response to health asset class questions, a wealth asset class activity based at least in part on the user's response to wealth asset class questions, and/or a purpose asset class activity based at least in part on the user's response to purpose asset class questions. At a subsequent time, the user may be presented with new questions retrieved from the library of questions and provided with new recommended activities based at least in part on the user input to each of the new questions and/or based at least in part on sensed or generated data associated with activities of the user. For example, every day the user may be presented with new questions and recommended activities.

While the user may be presented with first questions retrieved from the library of questions, further processing may occur to determine new questions for the user. A machine learning model may be generated and trained using different user inputs to result in the processing of user input data to identify questions that are more appropriate to be presented to the user at a subsequent time and/or to generate new questions on the fly to be presented to the user. As an example, user inputs responsive to the first questions may indicate a user preference to improve their sleeping habits and/or a user preference to not focus on eating healthy. For example, the remote system may determine, based at least in part on the user input data, that the user regularly answers in the affirmative to questions that are sleeping-related. Additionally, or alternatively, the remote system may determine, based at least in part on the user input data, that the user regularly answers in the negative to questions that are food related. Thus, the machine learning model may be trained such that user input data map to new questions to be presented to the user based at least in part on those user preferences. For example, a user who indicates a preference to improve sleeping habits may be presented with questions at subsequent times that are specific to sleeping. Additionally, or alternatively, a user who indicates a preference to not focus on eating healthy may not be presented with questions at subsequent times that are specific to eating.

In examples, user activity data may be received by the remote system when the user interacts with the user device and the application operating on the user device after being presented recommended activities. For example, the user may be presented with the recommended health asset class activity, the wealth asset class activity, and/or the purpose asset class activity. User activity inputs may indicate that the recommended activity has been completed, and/or request that another recommended activity be removed as a recommendation. A user may also request that a certain activity be added to the recommended activities. Additionally, or alternatively, the user device (or another remote computing device) may include sensor arrays that track the activity of the user while the user is performing the recommended activity and may provide the remote system with the user activity data indicating whether the user completed the recommended activity and/or did not attempt the recommended activity.

While the user may be presented with activities based at least in part on user inputs responsive to questions, further processing may occur to determine recommended activities for the user. For example, a machine learning model may be trained using different user activity inputs to result in the processing of user activity data to identify activities that are determined to be more appropriate to be presented to the user at a subsequent time. For example, user activity inputs responsive to recommended activities may indicate a user activity preference to play tennis and/or a user activity preference to not go to the bank. For example, the remote system may determine, based on the user activity data, that the user regularly completes the recommended activities that are tennis related. Additionally, or alternatively, the remote system may determine, based at least in part on the user activity data, that the user regularly requests the removal of bank-related activities as a recommendation. Thus, the machine learning model may be trained such that user activity data may map to new activities to be presented to the user based at least in part on those user activity preferences. For example, the user who indicates an activity preference to play tennis may be presented with activities at subsequent times that are specific to and/or similar to tennis (e.g., pickleball, squash, ping pong, badminton, etc.). Additionally, or alternatively, the user who indicates an activity preference to not go to the bank may not be presented with activities at subsequent times that are banking related.

In examples, the mood of the user may be used by the remote system to determine recommended activities. User input data representing the user input may be received by the remote system, where each question in the library of questions may be associated with the mood experienced by the user after the activity. Based on the user input data associated with mood experienced during the previous activity, the user may then be provided with recommended activities that are responsive to the user input and associated with the health, wealth, and/or purpose asset classes. For example, the user may be provided with a health asset class activity based at least in part on the user's response to previous mood experience questions. Additionally, the remote system may leverage challenges, routines, and notifications to encourage user participation and further inform recommended activities. Further, the remote system may categorize users based on various deficiencies, such as failure to complete an activity to name a nonlimiting example, to optimize activity recommendations.

In examples, the location of the user device may be used by the remote system to determine recommended activities that are determined to be more appropriate for the user (e.g., recommending an activity and/or entity that is in close proximity to the user device). For example, the user device may be equipped with capabilities for determining the location and/or geographic area of the device, such as a Global Position System (GPS). The user device (which may be described herein as a device and/or as a mobile device) may provide location information that indicates a location of the user using the application operating on the user device to the remote system. Based upon the location data, the remote system may generate a geofence including the location of the user device and a surrounding area centered upon the location of the user device and/or an area otherwise associated with the location of the user device. Additionally, or alternatively, the remote system may also receive the location and/or geographic area of an activity and/or entity. Based at least in part on the location data of the activity and/or entity, the remote system may generate a geofence including the location of the activity and/or entity and a surrounding area centered upon the location of the activity and/or entity and/or otherwise associated with the location of the activity and/or entity. The remote system may determine that the user and the activity and/or entity are in close proximity when the location of the activity and/or entity is within the geofence of the user device. For example, the remote system may determine that the location of a tennis court is in close proximity to the user based at least in part on the geographic location of a tennis court being within the user's geofence. The remote system may also determine that the location of a soccer field is not in close proximity to the user device based on the geographic location of the soccer field not being within the user's geofence. Thus, the remote system may determine that the more appropriate activity to recommend to the user is tennis. In another example, the remote system may determine that the location of a first emergency response entity is in close proximity to the user based at least in part on the geographic location of the first emergency response being within the user's geofence. The remote system may also determine that the location of a second emergency response entity is not in close proximity to the user device based at least in part on the geographic location of the second emergency response entity not being within the user's geofence.

In examples, user input data may be received by the remote system, where the user input data is associated with a threshold amount of action time. For example, the user input may indicate that the user is in distress and/or in a rush to engage in a given activity. Thus, there may be a low threshold amount of action time. Based at least in part on the threshold amount of action time, the user may then be provided with an indicator of a recommended activity and/or entity that is determined to be within the threshold amount of action time. Additionally, the remote system may provide the user with challenges, which allow the user to complete an activity or opportunity within a specified timeframe.

In examples, the remote system may create a Mood Index Score that scores the user in the three categories based at least on the users answers to questions from a library of questions. The Mood Index Score may be updated overtime as the user completes opportunities, activities, and answers questions. Based at least in part on this generated Mood Index Score, the remote system may provide recommended activities and opportunities to the user. Additionally, the remote system may prompt the user to complete a mood check-in each day and subsequently store the user input data created in response to the mood check-in. The remote system may use the stored user input data representing responses to the mood check-in, to override recommendations from the Mood Index.

In examples, the remote system may allow the use to set up and track routines that contain various tasks. The user may be allowed to adjust the number of times a task within a routine needs to be completed each day. For example, based at least in part on user input, the remote system may lower the number of times a user must complete a health activity, such as a walk. Additionally, the remote system may track user streaks for completing each routine. Further, the remote system may be configured to generate push notifications upon certain user input, action, or inaction. For example, a user may input into the remote system that they desire push notifications relating to recommended activities be provided every morning. In examples, the remote system may also allow users to log journal entries relating to various journal types, such as gratitude or daily reflection to name a few nonlimiting examples.

It should be noted that the exchange of data and/or information as described herein may be performed where a user has provided consent for the exchange of such information. For example, upon setup of user devices and/or initiation of applications, a user may be provided with the opportunity to opt in and/or opt out of data exchanges between user devices and/or for performance of the functionalities described herein. Additionally, when one of the user devices is associated with a first user account and another of the user devices is associated with a second user account, user consent may be obtained before performing some, any, or all of the operations and/or processes described herein. Additionally, the operations performed by the components of the systems described herein may be performed where a user has provided consent for performance of the operations.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting examples. The features illustrated or described in connection with one example may be combined with the features of other examples, including as between systems and methods. Such modifications and variations are intended to be included within the scope of the appended claims.

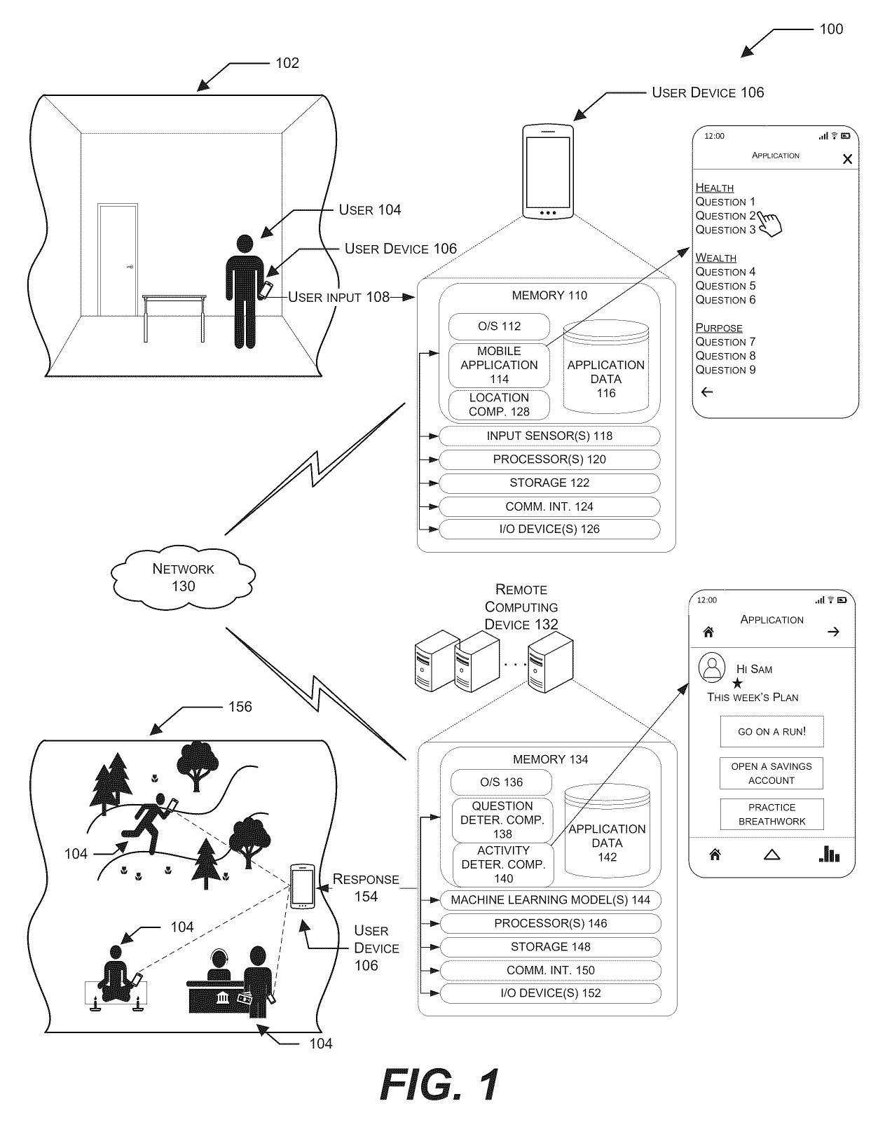

FIG. 1 illustrates a schematic diagram 100 of an illustrative environment 102 in which a user 104 is associated with a user device 106 in which user input 108 is detected by the user device 106, and a remote computing device 132 may perform processing on user input data representing the user input 108 to determine which questions and/or activities will respond to the user input 108 and be provided to the user 104.

The user device 106 may include at least one memory 110 and one or more processor(s) 120. The processor(s) 120 may be implemented as appropriate in hardware, computer-executable instructions, firmware, or combinations thereof. Computer-executable instruction or firmware implementations of the processor(s) 120 may include computer-executable or machine executable instructions written in any suitable programming language to perform the various functions described.

Memory 110 may include an operating system 112 and one or more application programs or services for implementing the features disclosed herein including at least a mobile application 114. The memory 110 may also include application data 116, which provides information to be generated by and/or consumed by the mobile application 114. In some examples, the application data 116 may be stored in a database. A mobile application may be any set of computer executable instructions installed upon, and executed from, a user device 106. In some examples, the mobile application 114 may cause the user device 106 to establish a communication session with remote computing device 132 that provides backend support for the mobile application 114. The remote computing device 132 may maintain account information associated with the user 104. In examples, the user 104 may log into the mobile application 114 in order to access functionality provided by the mobile application 114.

In examples, the user 104 in environment 102 may desire to create a personal wellness plan and may interact with the user device 106. For example, the user device 106 may receive user input 108 from user 104, indicating a request to create the personal wellness plan as part of making a user account on a mobile application 114 installed on the user device 106. The user device 106 may include one or more input sensors 118 for receiving user input 108 and/or user activity input. There may be a variety of input sensors 118 capable to detecting user input 108 and/or user activity input, such as an accelerometer, a camera, a microphone, a global position system (e.g., GPS) receiver, etc.

The user device 106 may also contain communications interface(s) 124 that enable the user device 106 to communicate with any other suitable electronic devices. In some examples, the communication interface 124 may enable the user device 106 to communicate with other electronic devices on the network 130. For example, the user device 106 may include a Bluetooth, Wi-Fi, Cellular, LTE, etc. communication module(s), which allows the user device 804 to communicate with another electronic device. The user device 106 may also include input/output (I/O) device(s) 126, such as for enabling connection with a keyboard, a mouse, a pen, a voice input device, a touch input device, a display, speakers, a printer, etc.

The user device 106 may also include storage 122, such as either removable storage or non-removable storage including, but not limited to, magnetic storage, optical disks, and/or tape storage. The disk drives and their associated computer-readable media may provide non-volatile storage of computer-readable instructions (including non-transitory computer-readable instructions), data structures, program modules, and other data for the computing devices. In some implementations, the memory 110 may include multiple different types of memory, such as static random-access memory (SRAM), dynamic random-access memory (DRAM), or ROM.

The memory 110 may store program instructions that are loadable and executable on the processor(s) 120, as well as data generated during the execution of these programs. Depending on the configuration and type of user device 106, the memory 110 may be volatile (such as random-access memory (RAM)), non-volatile (such as read-only memory (ROM), flash memory, miniature hard drive, memory card, etc.), or some combination thereof. In at least one example, the memory 110 of user device 106 may include at least one component for performing various functions as described herein.

In some examples, the user device 106 may communicate with the remote computing device 132 via the network 130. The network 130 may include any one or a combination of many different types of networks, such as cable networks, the Internet, wireless networks, cellular networks, and other private and/or public networks. In addition, the network 130 may comprise multiple different networks. For example, the user device 106 may utilize a wireless local area network (WLAN) to communicate with a wireless router, which may then route the communication over a public network (e.g., the Internet) to the remote computing device 132. For example, when the user device 106 receives user input 108 indicating a request to make a personal wellness plan, the user device 106 may communicate user input data to the remote computing device 132.

The remote computing device 132 may be any computing device configured to perform one or more calculations on behalf of the mobile application 114 on the user device 106. In an example, the remote computing device 132 may include at least one memory 134 and one or more processor(s) 146. The processor(s) 146 may be implemented as appropriate in hardware, computer-executable instructions, firmware, or combinations thereof. Computer-executable instruction or firmware implementations of the processor(s) 146 may include computer-executable or machine executable instructions written in any suitable programming language to perform the various functions described. The memory 134 may include an operating system 136, application data 142, and a question determination component 138 and activity determination component 140 for providing recommended questions and activities to the user device 106. The question determination component 138 may receive data from the user device 106, such as user input data, and may determine questions to display, from a library of questions, on the user device 106 to the user 104. Additionally, or alternatively, the activity determination component 140 may receive data from the user device 106, such as user activity data, and may determine recommended activities to display, from a library of activities, on the user device 106 to the user 104.

The memory 134 and the storage 148, both removable and non-removable, are examples of computer-readable storage media. For example, computer-readable storage media may include volatile or non-volatile, removable or non-removable media implemented in any method or technology for storage of information such as computer-readable instructions, data structures, program modules or other data. The remote computing device 132 may also contain communications interface(s) 150 that allow the remote computing device 132 to communicate with a stored database, another computing device or server, user terminals, and/or other components of the described system. The remote computing device 132 may also include input/output (I/O) device(s) 152, such as for enabling connection with a keyboard, a mouse, a pen, a voice input device, a touch input device, a display, speakers, a printer, etc.

The memory 134 may store program instructions that are loadable and executable on the processor(s) 146, as well as data generated during the execution of these programs. Depending on the configuration and type of remote computing device 132, the memory 134 may be volatile (such as random-access memory (RAM)) and/or non-volatile (such as read-only memory (ROM), flash memory, etc.). The remote computing device 132 may also include storage 148, such as either removable storage or non-removable storage including, but not limited to, magnetic storage, optical disks, and/or tape storage. The disk drives and their associated computer-readable media may provide non-volatile storage of computer-readable instructions, data structures, program modules, and other data for the computing devices. In some implementations, the memory 134 may include multiple different types of memory, such as static random-access memory (SRAM), dynamic random-access memory (DRAM), or ROM.

In some examples, the remote computing device 132 may determine questions to be presented to the user 104 via the user device 106 based on receiving the user input data indicating a request to make a personal wellness plan. The question determination component 138 of the remote computing device 132 may store a library of questions. The remote computing device 132 may associate the questions from the library of questions with specific asset classes such as health, wealth, and/or purpose. For example, questions pertaining to exercise and/or food may be associated with the health asset class. Questions pertaining to investment and/or savings goals may be associated with the wealth asset class. Questions pertaining to mental health and/or personal relationships may be associated with the purpose asset class.

The remote computing device 132 may provide question determination data, indicating at least one question associated with each of the health, wealth, and/or purpose asset classes to the user device 106 for display. For example, the user may be presented with nine questions: three questions from the health asset class; three questions from the wealth asset class; and three questions from the purpose asset class. The user may then provide user input 108 (e.g., responses) to each of the questions, where the user input 108 is detected by the input sensor(s) 118 of the user device 106.

When the user device 106 receives user input 108 indicating responses to each of the questions, the user device 106 may communicate the user input data representing the responses to the remote computing device 132. In some examples, the remote computing device 132 may determine activities to be recommended to the user 104 via the user device 106 based on receiving the user input data indicating responses to each of the questions. The activity determination component 140 of the remote computing device 132 may store a library of activities. For example, activities associated with the health asset class may include running or biking. Activities associated with the wealth asset class may include opening a savings account or starting a budget plan. Activities associated with the purpose asset class may include learning about breath work or watching a video on meditation. The remote computing device 132 may associate the activities from the library of activities with the specific asset classes such as health, wealth, and/or purpose. Additionally, or alternatively, the remote computing device 132 may associate each question in the library of questions with one or more activities from the library of activities.

In an example, based on the user input data representing the responses to each of the questions determined to be presented by the question determination component 138, the activity determination component 140 may determine the appropriate activities to be presented to the user 104. For example, the user may be provided with a health asset class activity based upon the user's response to health asset class questions, a wealth asset class activity based upon the user's response to wealth asset class questions, and/or a purpose asset class activity based upon the user's response to purpose asset class questions. The remote computing device 132 may provide activity determination data to the user device 106 for display, indicating at least one activity associated with health, wealth, and/or purpose asset classes. At a subsequent time, the user may be presented with new questions retrieved from the library of questions and/or then provided with new recommended activities based on the user input to each of the new questions. For example, every day the user may be presented with new questions and recommended activities.

The remote computing device 132 may also include on or more machine learning model(s) 144. The machine learning model(s) 144 may be configured to determine questions to be presented to the user 104 and/or activities to be recommended to the user 104. In examples, the machine learning model(s) 144 may be associated with, for example, decision tree learning, association rule learning, artificial neural networks (including, in examples, deep learning), inductive logic programming, support vector machines, clustering, Bayesian networks, reinforcement learning, representation learning, similarity and metric learning, sparse dictionary learning, and/or rules-based artificial intelligence.

In examples, one or more of machine learning model(s) 144 may be utilized to perform one or more operations described herein, including determining questions to be presented to the user 104 and/or activities to be recommended to the user 104. In these examples, the machine learning model(s) 144 may be generated and configured to intake the historical data and to generate, as output, the determinations described herein. A training dataset representing feedback data that indicates performance of the machine learning model(s) 144 may be generated and may be utilized to train the machine learning model(s) 144. For example, the feedback data may include any data indicating that a certain user input is associated with a certain questions and/or activity and may be utilized to determine subsequent questions and/or activities to present to a user. Generation of the training dataset may include formatting the feedback data into input vectors for the artificial intelligence model to intake, as well as associating the various data with the outcomes of the questions and/or activities described herein. Generation of the trained artificial intelligence models may include updating parameters and/or weightings and/or thresholds utilized by the models to determine appropriate questions to present to the user, appropriate activities to recommend, and the like. These trained machine learning models 144 may then be stored in association with the user device 106 and/or the account of user 104 and may be utilized to subsequently determine the results described above.

For example, once the user input data is acquired by the remote computing device 132, analysis may be performed on the user input data utilizing machine learning model(s) 144 trained using different user inputs to identify questions that are more appropriate to be presented to the user 104 at a subsequent time. User input 108 responsive to questions generated by the question determination component 138 may indicate a preference of user 104 to improve their sleeping habits and/or a preference of user 104 to not focus on eating healthy. For example, the question determination component 138 may determine, based on user input data, that the user 104 regularly answers in the affirmative to questions that are sleeping related. Additionally, or alternatively, the question determination component 138 may determine, based on the user input data, that the user 104 regularly answers in the negative to questions that are food related. With consent of users, application data 142 may include data indicating these preferences of the user 104, and the question determination component 138 may determine subsequent questions to display on the user device 106 that are in accordance with the preferences of user 104. For example, the question determination component 138 may provide instructions to the user device 106 to update the questions displayed on the user device 106.

The activity determination component 140 may receive data from the user device 106, such as user activity data, and may determine subsequent activities to recommend on the user device 106 to the user 104 (e.g., response 154). User input 108 may indicate that a previously recommended activity has been completed, and/or request that another recommended activity be removed as a recommendation. User input 108 may also request that a certain activity be added to the recommended activities. Input sensors(s) 118 associated with the user device 106 may provide the remote computing device 132 with the user activity data indicating whether the user completed the recommended activity and/or did not attempt the recommended activity, such as in illustrative environment 156.

Once the user activity data is acquired by the remote computing device 132, analysis may be performed on the user activity data utilizing machine learning model(s) 144 trained using different user inputs to identify activities that are more appropriate to be presented to the user 104 at a subsequent time. As an example, user input 108 responsive to recommended activities previously generated by the activity determination component 140 may indicate an activity preference of user 104 of tennis and/or an activity preference of user 104 to not go to the bank. For example, the activity determination component 140 may determine, based on user activity data, that the user 104 regularly completes the recommended activities that are tennis related. Additionally, or alternatively, the activity determination component 140 may determine, based on user activity data, that the user 104 regularly requests the removal of bank-related activities as a recommendation. With the consent of users, application data 142 may include data indicating these activity preferences of the user 104, and the activity determination component 140 may determine subsequent activities to recommend on the user device 106 that are in accordance with the activity preferences of user 104. For example, the activity determination component 140 may provide instructions to the user device 106 to update the recommended activities in the personal wellness plan accessed from the application data 142.

The applications or other components described herein may be configured to execute in the foreground and background of the user device 106. For example, the mobile application 114 may be configured to execute in the foreground when the user 104 is actively engaged in one or more of the functionalities of the mobile application 114. In other examples, the mobile application 114 may be configured to execute in the background when the user 104 is not actively engaged in on or more of the functionalities, but the mobile application 114 is still “open” and capable of communicating with other applications on the user device 106. The mobile application 114, running in the background, may be caused to be displayed in the foreground in response to selection of certain functionality on one or more applications utilized by the user device 106. It should also be understood that the mobile application 114, or the functionality associated therewith, can be integrated with other applications, such as third-party applications.

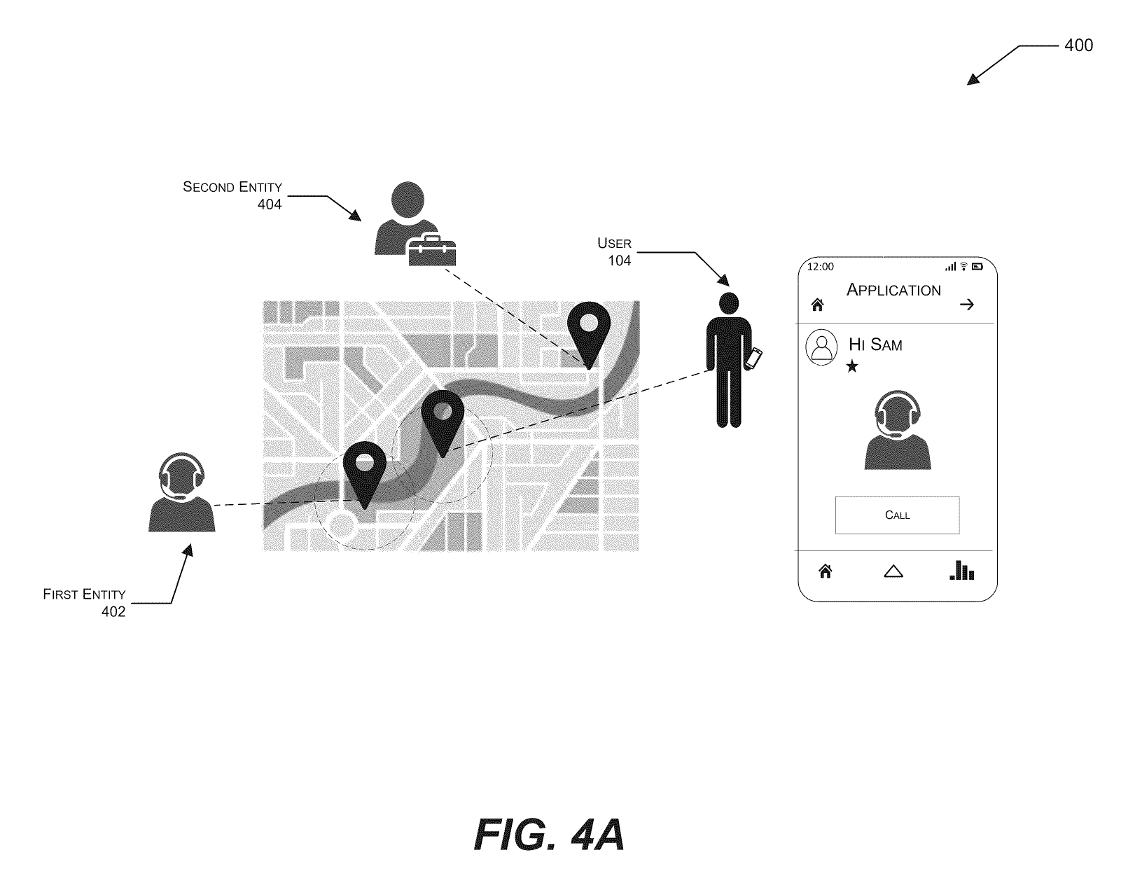

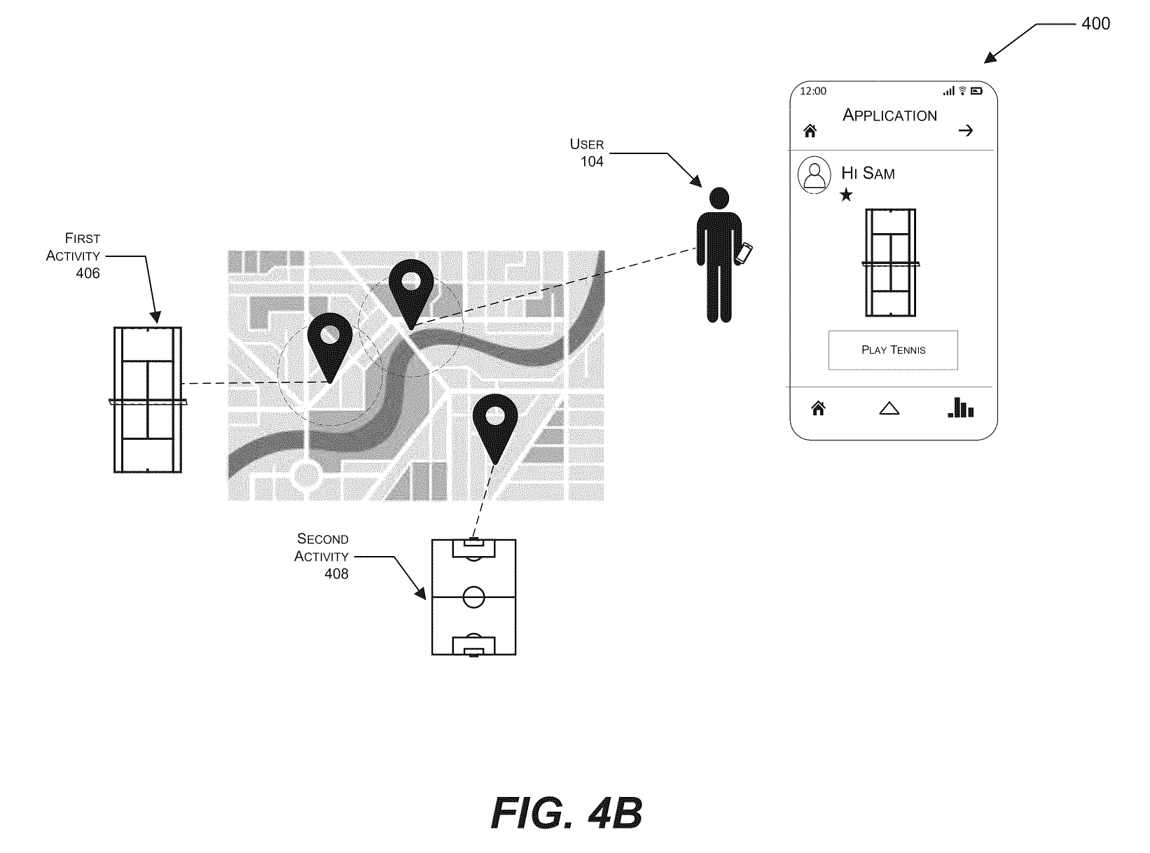

In an example, the location component 128 of the user device 106 can be used to identify a location of the user 104. In at least one example, the location of the user 104 may be used by the activity determination component 140, described above, to determine recommended activities that are more appropriate for the user 104. That is, in some examples, the activity determination component can implement geofencing to determine particular activities for the user 104. Additionally, or alternatively, user input data indicating a threshold amount of time to complete an activity may be used by the activity determination component 140 along with geofencing to determine proximate activities for the user 104. Additional details associated with the use of geofencing are described below with reference to FIGS. 4A and 4B.

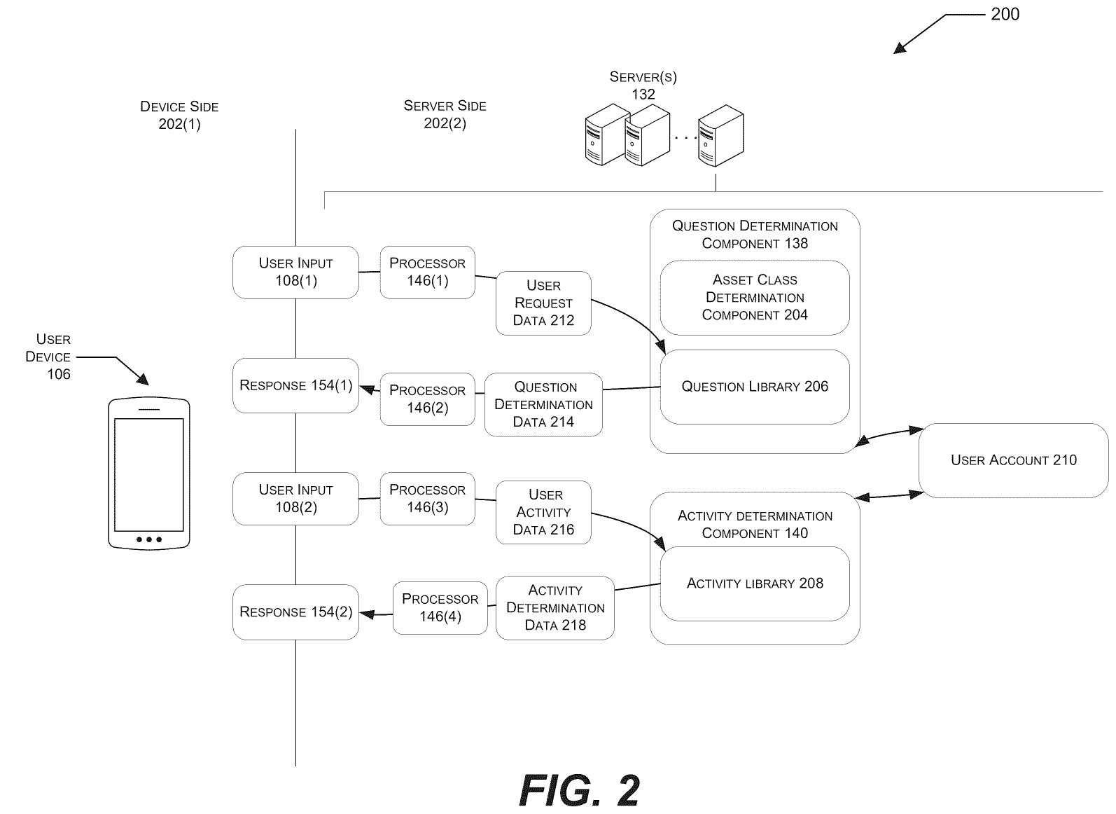

FIG. 2 illustrates example components of the remote computing device 132 of FIG. 1 that performs an example of processing user input data representing the user input 108 of the user 104 to determine questions to be presented and/or activities to be recommended to the user 104. As illustrated, FIG. 2 is split into a device-side 202(1), corresponding to the environment 102, and a server side 202(2), corresponding to the remote computing device 132. Other user devices may be substituted for the illustrated devices or added to the device side 202(1).

As shown, the user device 106 may receive user input 108(1). The user device 106 may transmit the user input 108(1) (e.g., a request to create an account and/or personal wellness plan) from the device side 202(1) to the remote computing device 132 on the server side 202(2). The processor 146(1) of the remote computing device 132 may convert the user input 108(1) into user request data 212, where the user request data 212 corresponds to the user input 108(1). The question determination component 138 may provide the user request data 212 to a question library 206. The question library 206 determines whether a certain question should be selected to be presented to the user 104. For example, the question library 206 may determine which questions are associated with a health, wealth, and/or purpose asset class. The asset class determination component 204 may select questions that are associated with each type of asset class (e.g., at least one question associated with the health asset class, one question associated with the wealth asset class, and one question associated with the purpose asset class), and the question determination data 214 may be received by the processor 146(2). The question determination component 138 may also query the user account 210 to determine questions that have previously been provided to the user 104.

The processor 146(2) may generate a response 154(1) for the user device 106 to perform. For example, the response 154(1) may be a display of the questions selected by the question determination component 138. Additionally, or alternatively, the user device 106 may receive user input 108(2). The user device 106 may transmit the user input 108(2) (e.g., a response to the displayed questions) from the device side 202(1) to the remote computing device 132 on the server side 202(2). The processor 146(3) of the remote computing device 132 may convert the user input 108(2) into user input data 216, where the user input data 216 corresponds to the user input 108(2).

The activity determination component 140 may provide the user input data 216 to an activity library 208. The activity library 208 determines whether a certain activity should be recommended to the user 104. For example, the activity library 208 may determine which activities are most appropriate for the user 104 based on the user input data 216. The activity determination component 140 may then select activities that are associated with the user input 108(2), and the activity determination data 218 may be received by the processor 146(4). The activity determination component may also query the user account 210 to determine activities that have previously been recommended to the user 104. The processor 146(4) may generate a response 154(2) for the user device 106 to perform. For example, the response 154(2) may be a display of the recommended activities selected by the activity determination component 140.

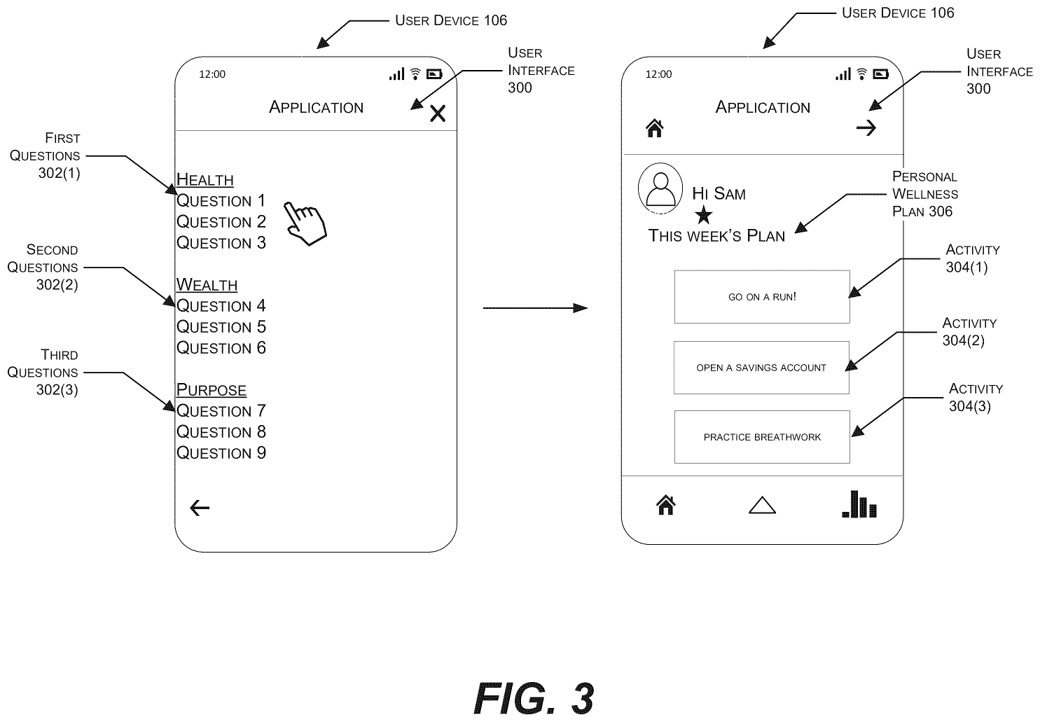

FIG. 3 illustrates an example user interface 300 for the display of questions and recommended activities. For example, the user interface 300 may be displayed on user device 106 associated with user 104. User input may be received via the user interface and may be utilized to select one or more activities to be recommended to the user 104. For example, first questions 302(1), second questions 302(2), and/or third questions 302(3) may be displayed via the user interface 300. In this example, first questions 302(1) are associated with the health asset class, second questions 302(2) are associated with the wealth asset class, and/or third questions 302(3) are associated with the purpose asset class. As shown in FIG. 3, user input is received indicating selected response to each question (e.g., “sometimes,” “always,” “never,” etc.).

Once the user 104 answers the questions, the user interface may display recommended activity 304(1), 304(2), and/or 304(3) as part of the personal wellness plan 306. For example, activity 304(1) may be associated with the health asset class and recommends an activity such as “go on a run!” Activity 304(2) may be associated with the wealth asset class and recommends an activity such as “open a savings account.” Activity 304(3) may be associated with the purpose asset class and recommends an activity such as “practice breathwork.”

The user interface 300, in response to user input 108, may display only relevant elements based on the answers provided to the first questions 302(1), second questions 302(2), and third questions 302(3). Further, the user interface 300 may display relevant elements based on sensor data gathered by an input sensor 118 (e.g., an accelerometer, camera, microphone, or GPS receiver). The processors 146(2) and 146(4) may filter elements to be displayed on the user interface 300 to reduce the overall amount of information on a screen of limited size. For example, a user 104 may answer a question stating that their preferred health asset class was “biking,” the user interface 300 may then display “biking” as an option for a health asset class. Additionally, the user interface 300 may display “running” based on sensor data gathered by an input sensor 118 such as an accelerometer collecting movement data associated with the running motion.

The user interface 300 may generate user interface elements such as, but not limited to, examples displayed in FIG. 3. Generation of interface elements may be in response to receiving application data 116 from a user 104 answering the first questions 302(1), second questions 302(2), and third questions 302(3). Content appearing in the interface elements may be determined by user input 108(1) and 108(2) and/or by a machine learning model trained to recommend a user's preferred setting mode of activity. The user interface 300 may show other elements such as, but not limited to, user profile data, hyperlinks, and/or navigational buttons.

The mobile application 114 may collect application data 116 by way of secure and encrypted communications between the mobile application 114 and other applications stored on the user device 106 to be displayed on the user interface 300. For example, the application data 116 may indicate that a health asset class “hiking” should be recommended based on another application's preference for recommending “hiking” to the user 104. This collection of application data allows for dynamic updates to the user interface 300 based on the most relevant data stored on the user device 106.

Additionally, the mobile application 114 may bring to the foreground of the user device 106 the user interface 300 in response to a trigger event (e.g., the completion of activity). For example, a user 104 may complete activity 304(1) “go on a run!” based on data collected by an input sensor 118 such as GPS data within a time period. The mobile application 114 may then display the user interface in the foreground of the user device 106 automatically, wherein the trigger event was the completion of activity 304(1).

The user interface 300 may include hyperlinks that a user 104 may select. Hyperlinks displayed on the user interface 300 may be generated based on recommended activities in response to receiving application data 116. A user 104 may select the hyperlinks causing the user interface 300 to display different content (e.g., transition to a different user interface or opening another application). For example, a user 104 may select activity 304(1) causing the user interface 300 to display a screen containing information about the run that the user 104 may choose to complete. Additionally, or alternatively, a user 104 may select activity 304(1) causing the mobile application 114 to open another application that uses GPS to track a user's activity and record activity data.

In addition to the above, the user interface 300, commanded by the processors 146 and in response to various user inputs 108, may filter elements displayed on the user interface 300 to reduce the overall amount of information on a screen of limited size. For example, a user 104 may indicate that certain user interface elements, such as answers to the first questions 302(1), are not relevant and therefore do not need to be displayed on the user interface 300. Further, a user 104 may indicate that certain user interface elements, such as the personal wellness plan 306, are relevant and therefore do need to be displayed on the user interface 300. Additionally, the user interface 300, in response to various user inputs 108, may reduce/filter the display of interface elements based on sensor data gathered by an input sensor 118 (e.g., a camera or GPS receiver). For example, the user interface 300, in response to user input 108, may determine that the interface elements relating to GPS sensor data are not relevant to the user 104 and therefore should not be displayed on the user interface 300. Further, the user interface 300, in response to user input 108, may determine that the user interface elements relating to sensor data from an accelerometer are relevant to the user 104 and therefore should display the accelerometer interface elements on the user interface 300.

Additionally, the mobile application 114 may be configured to generate interactive elements associated with the functionality of data objects. Such interactive elements may be displayed on the user interface 300 where they are then usable by the user 104. For example, the generated interactive elements may prompt the user 104 to provide particular preference data, such as inputs to customize audible alerts and notifications, as a few nonlimiting examples. The interactive elements may be used to generate data that associates entities, audible alerts, trigger events, and the like with each other. Additionally, the interactive elements may prompt the mobile application 114 to create, generate, or display user interface elements on the user interface 300 in response to user inputs 108, such as generated hyperlinks or push notifications, to name a few nonlimiting examples.

By way of secured and encrypted communications between the mobile application 114 and other applications stored on the user device 106, the user device 106 may dynamically determine, based on acquired data (e.g., a question response or an input sensor data), the user interface elements to be displayed on the user interface 300. For example, the user device 106 may communicate, via a secured and encrypted manner, with the application data 166 and determine that an interface element representing the health asset class “hiking” should be displayed on the user interface 300 based on a dynamic comparison to another application's data (e.g., a question's response indicating “hiking” as a preferred health asset). The secured and encrypted dynamic communication between various applications stored on the user device 106, may allow for updates to the user interface 300 based on the most relevant data stored on the user device 106. The dynamic determination of which user interface elements are to be displayed on the user interface 300 may allow the user device 106 to provide the most appropriate and relevant information in order to ensure a more positive and effective user 104 experience.

The mobile application 114 may, based at least in part on a trigger event (e.g., the completion of an activity or the collection of certain sensor data), modify the position of a user interface element(s) displayed on the user device 106. For example, a user 104 may be prompted to complete activity 304(1) “go on a run!”. Based on data collected by an input sensor 118, such as GPS data collected within a time period, the mobile application 114 may then display the associated user interface element in the foreground of the user device 106 automatically (i.e., the trigger event being the calculated completion of activity 304(1)). Further, the mobile application 114 may, automatically and without user input 108, move any of the various user interface elements (e.g., questions or activities, as a few nonlimiting examples) either to or from the foreground of the user device 106, based at least in part on some trigger event happening. The mobile application's 144 automatic management of user interface elements may allow for a more positive user 104 experience and may eliminate the amount of user inputs 108 needed from the user 104 in order for the mobile application 114 to operate efficiently and effectively.

In some examples, the user interface 300 may generate, in response to certain trigger events, hyperlinks that may be displayed on the user device 106 which the user 104 may interact with. The hyperlinks displayed on the user interface 300 may be generated based at least in part on various inputs received by the mobile application 114, such as user inputs 108 or sensor inputs, to name a few nonlimiting examples. Additionally, the mobile application 114 may automatically generate hyperlinks in response to dynamic secured and encrypted communications between the mobile application 114 and other applications stored on the user device, such as gathered input sensor data triggering the completion of a particular event and the generation of a relevant hyperlink. Further, the user 104 may prompt the generation of hyperlinks by interacting with the appropriate user interface elements and/or interactive elements, such as creating a hyperlink to additional “hiking” resources in response to user inputs 108, as a nonlimiting example. When selected, the hyperlinks displayed on the user interface 300 may cause an array of responses, such as transitioning to a different user interface, opening another application, or providing additional resources relating to a particular input, to name a few nonlimiting examples. The use of hyperlinks may allow for greater user 104 interaction, experience, and satisfaction when interacting with the mobile application 114 and its user interface elements.

FIGS. 4A and 4B illustrate an example environment 400 for determining nearby entities and/or activities based on the geographic locations of the entities and/or activities between within a geofence that is generated based on the location data of user 104. As mentioned with respect to FIG. 1, the user device 106 may be equipped with a sensor such as a GPS component configured to identify a geographic location of the user device 106. Once the user device 106 determines its location, it may then provide the location data to the remote computing device 132. Based on the location data, the remote computing device 132 may determine a dynamic geofence 406 that is centered around the location of the user 104 and includes an additional area surrounding the location of user 104. The geofence 406 may be updated to reflect any changes to the location of the user 104 (e.g., when the user 104 is moving around). Additionally, or alternatively, the remote system may also receive the location and/or geographic area of an activity and/or entity.

In the example of FIG. 4A, in response to receiving the location data of user 104, the remote computing device 132 may determine that the location of second entity 404 is not within the geofence 406, and thus may determine that the second entity 404 is not in proximity to the user 104. Additionally, or alternatively, the remote computing device 132 may determine that the location of the first entity 402 is within the geofence 406, and thus may determine that the first entity 402 is in proximity to the user 104. Because the first entity 402 is in proximity to user 104 based on geofence 406, the remote computing device 132 may then determine that the first entity 402 should be recommended to the user. For example, when user input data may indicate that the user 104 is in distress, the remote computing device 132 may determine an emergency response entity that is in close proximity to the user 104, and cause information associated with the emergency response entity to be displayed on the user device 106.

In the example of FIG. 4B, in response to receiving the location data of user 104, the remote computing device 132 may determine that the location of second activity 404 is not within the geofence 406, and thus may determine that the second activity 404 is not in proximity to the user 104. Additionally, or alternatively, the remote computing device 132 may determine that the location of the first activity 402 is within the geofence 406, and thus may determine that the first activity 402 is in proximity to the user 104. Because the first activity 402 is in proximity to user 104 based on geofence 406, the remote computing device 132 may then determine that the first activity should be recommended to the user. For example, the remote computing device 132 may determine that the location of a tennis court is in close proximity to the user 104 based on the geographic location of a tennis court being within the geofence 406. The remote computing device 132 may also determine that the location of a soccer field is not in close proximity to the user 104 based on the geographic location of the soccer field not being within the geofence 406. Thus, the remote computing device 132 may determine that the more appropriate activity to recommend to the user is tennis.

In another example, user input data may be received by the remote computing device, where the user input data is associated with a threshold amount of action time. For example, when user input data may indicate that the user 104 is in distress and may have a low threshold of action time, the remote computing device 132 may determine an entity (e.g., the first entity 402) that would be accessible within that threshold amount of time. Additionally, or alternatively, when user input data may indicate that the user 104 is in a rush to engage in an activity and may have a low threshold of action time, the remote computing device 132 may determine a sports court that is in close proximity to the user 104 and within the threshold amount of action time, and cause information associated with sport court to be displayed on the user device 106.

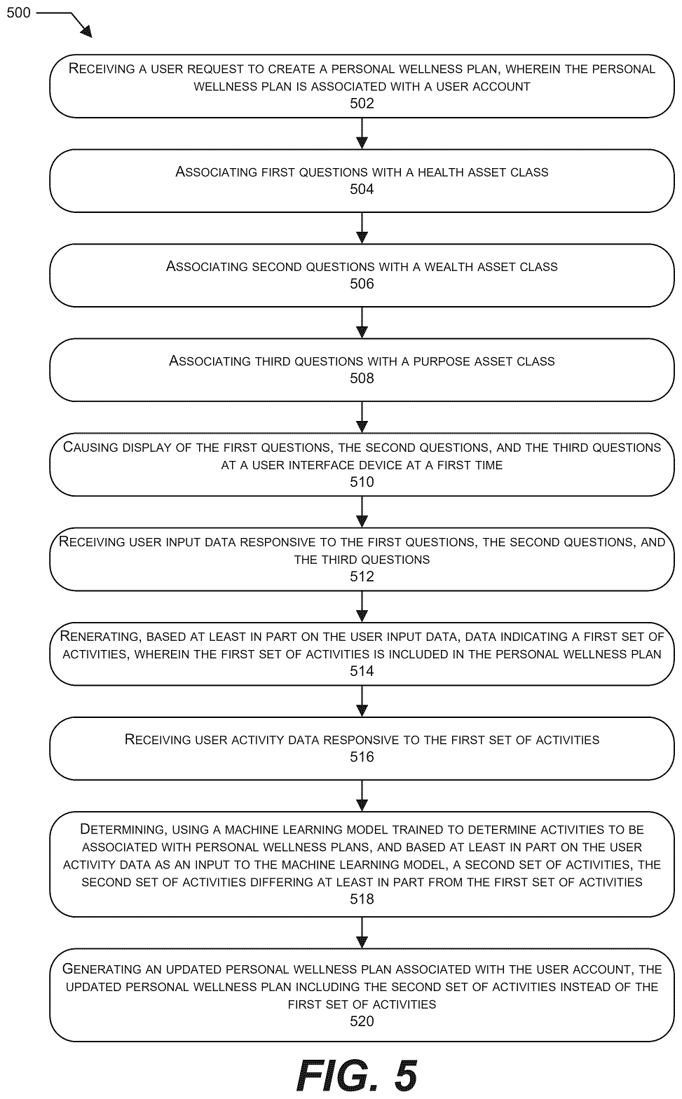

FIGS. 5-7 are example processes for dynamic activity recommendation output. The processes described herein are illustrated as collections of blocks in logical flow diagrams, which represent a sequence of operations, some or all of which may be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks may be executed. For discussion purposes, the processes are described with reference to the environments, architectures and systems described in the examples herein, such as, for example those described with respect to FIGS. 1-4, although the processes may be implemented in a wide variety of other environments, architectures, and systems.

FIG. 5 is a flow diagram of an example process 500 for dynamic activity recommendation output according to an example described herein. The order in which the operations or steps are described is not intended to be construed as a limitation, and any number of the described operations may be combined in any order and/or in parallel to implement process 500.

At block 502, the process 500 may include receiving a user request to create a personal wellness plan, wherein the personal wellness plan is associated with a user account. For example, a user may desire to create a personal wellness plan and may interact with a user device. For example, the user device may receive user input from the user, indicating a request to create the personal wellness plan as part of making a user account on a mobile application installed on the user device. The user device may include one or more input sensors for receiving user input and/or user activity input. There may be a variety of input sensors capable to detecting user input and/or user activity input, such as an accelerometer, a camara, a microphone, a global position system (e.g., GPS) receiver, etc. In some examples, the user device may communicate with a remote computing device via a network. The network may include any one or a combination of many different types of networks, such as cable networks, the Internet, wireless networks, cellular networks, and other private and/or public networks. In addition, the network may comprise multiple different networks. For example, the user device may utilize a wireless local area network (WLAN) to communicate with a wireless router, which may then route the communication over a public network (e.g., the Internet) to the remote computing device. For example, when the user device receives user input indicating a request to make a personal wellness plan, the user device 106 may communicate user input data to the remote computing device.

At block 504, the process 500 may include associating first questions with a health asset class. In an example, the remote computing device may include at least one memory and one or more processor(s). The memory may include an operating system, application data, and a question determination component for providing recommended questions to the user device. In some examples, the remote computing device may determine questions to be presented to the user via the user device based on receiving the user input data indicating a request to make a personal wellness plan. The question determination component of the remote computing device may store a library of questions. The remote computing device may associate the questions from the library of questions with specific asset classes such as health. For example, questions pertaining to exercise and/or food may be associated with the health asset class.

At block 506, the process 500 may include associating second questions with a wealth asset class. For example, the remote computing device may determine questions to be presented to the user via the user device based on receiving the user input data indicating a request to make a personal wellness plan. The question determination component of the remote computing device may store a library of questions. The remote computing device may associate the questions from the library of questions with specific asset classes such as wealth. For example, questions pertaining to investment and/or savings goals may be associated with the wealth asset class.

At block 508, the process 500 may include associating third questions with a purpose asset class. For example, the remote computing device may determine questions to be presented to the user via the user device based on receiving the user input data indicating a request to make a personal wellness plan. The question determination component of the remote computing device may store a library of questions. The remote computing device may associate the questions from the library of questions with specific asset classes such as purpose. For example, questions pertaining to mental health and/or personal relationships may be associated with the purpose asset class.

At block 510, the process 500 may include causing display of the first questions, the second questions, and the third questions at a user interface device at a first time. For example, the question determination component may receive data from the user device, such as user input data, and may determine questions to display, from a library of questions, on the user device to the user. A remote computing device may provide at least one question associated with health, wealth, and/or purpose asset classes to a user device for display on a mobile application. For example, the user may be presented with nine questions: three questions from the health asset class; three questions from the wealth asset class; and three questions from the purpose asset class.

At block 512, the process 500 may include receiving user input data responsive to the first questions, the second questions, and the third questions. For example, the user may provide user input (e.g., responses) to each of the questions, where the user input is detected by the input sensor(s) of the user device. When the user device receives user input indicating responses to each of the questions, the user device may communicate the user input data representing the responses to the remote computing device.

At block 514, the process 500 may include generating, based at least in part on the user input data, data indicating a first set of activities, wherein the first set of activities is included in the personal wellness plan. For example, the memory associated with the remote computing device may include an activity determination component for providing recommended activities to the user device. The activity determination component may receive data from the user device, such as user input data, and may determine recommended activities to display, from a library of activities, on the user device to the user.

In some examples, the remote computing device may determine activities to be recommended to the user via the user device based on receiving the user input data indicating responses to each of the questions. The activity determination component of the remote computing device may store the library of activities. For example, activities associated with the health asset class may include running or biking. Activities associated with the wealth asset class may include opening a savings account or starting a budget plan. Activities associated with the purpose asset class may include learning about breath work or watching a video on meditation. The remote computing device may associate the activities from the library of activities with the specific asset classes such as health, wealth, and/or purpose. Additionally, or alternatively, the remote computing device may associate each question in the library of questions with one or more activities from the library of activities.

In an example, based on the user input data representing the responses to each of the questions determined to be presented by the question determination component, the activity determination component may determine the appropriate activities to be presented to the user. For example, the user may be provided with a health asset class activity based upon the user's response to health asset class questions, a wealth asset class activity based upon the user's response to wealth asset class questions, and/or a purpose asset class activity based upon the user's response to purpose asset class questions. The remote computing device may provide activity determination data to the user device for display, indicating at least one activity associated with health, wealth, and/or purpose asset classes.

At block 516, the process 500 may include receiving user activity data responsive to the first set of activities. For example, the activity determination component of the remote computing device may receive data from the user device, such as user activity data. User activity data may indicate that a previously recommended activity has been completed, and/or request that another recommended activity be removed as a recommendation. User activity data may also indicate a user request that a certain activity be added to the recommended activities. Input sensors associated with the user device may provide the remote computing device with the user activity data indicating whether the user completed the recommended activity and/or did not attempt the recommended activity.

At block 518, the process 500 may include determining, using a machine learning model trained to determine activities to be associated with personal wellness plans, and based at least in part on the user activity data as an input to the machine learning model, a second set of activities, the second set of activities differing at least in part from the first set of activities. For example, one or more of machine learning model(s) may be utilized to perform one or more operations described herein, including determining activities to be recommended to the user. Once the user activity data is acquired by the remote computing device, analysis may be performed on the user activity data utilizing machine learning model(s) trained using different user inputs to identify activities that are more appropriate to be presented to the user at a subsequent time. As an example, user input responsive to recommended activities previously generated by the activity determination component may indicate an activity preference of the user of tennis and/or an activity preference of user to not go to the bank. For example, the activity determination component may determine, based on user activity data, that the user regularly completes the recommended activities that are tennis related. Additionally, or alternatively, the activity determination component may determine, based on user activity data, that the user regularly requests the removal of bank-related activities as a recommendation. With the consent of users, application data may include data indicating these activity preferences of the user, and the activity determination component may determine subsequent activities to recommend on the user device that are in accordance with the activity preferences of user. For example, the activity determination component may provide instructions to the user device to update the recommended activities in the personal wellness plan accessed from the application data.

At block 520, the process 500 may include generating an updated personal wellness plan associated with the user account, the updated personal wellness plan including the second set of activities instead of the first set of activities. For example, the activity determination component may provide instructions to the user device to update the recommended activities in the personal wellness plan accessed from the application data.

Additionally, or alternatively, the process 500 may include, wherein the user input data is first user input data, and the user activity data is first user activity data, associating fourth questions with the health asset class, associating fifth questions with the wealth asset class, and associating sixth questions with the purpose asset class. The process 500 may also include causing display of the fourth questions, the fifth questions, and the sixth questions at the user interface device at a second time. The process 500 may also include receiving second user input data responsive to the fourth questions, the fifth questions, and the sixth questions and generating, based at least in part on the second user input data, data indicating a third set of activities, wherein the third set of activities is included in the personal wellness plan. The process 500 may also include receiving second user activity data responsive to the third set of activities and determining, using the machine learning model trained to determine activities to be associated with personal wellness plans, and based at least in part on the second user activity data as the input to the machine learning model, a fourth set of activities, the fourth set of activities differing at least in part from the third set of activities. The process 500 may also include generating an updated personal wellness plan associated with the user account, the updated personal wellness plan including the fourth set of activities instead of the third set of activities.

Additionally, or alternatively, the process 500 may include receiving first user location data associated with the user account and generating, based at least in part on the first user location data, a geofenced area. The process 500 may also include receiving first action location data associated with a first emergency response entity, selecting the first emergency response entity based in part on the first user location data and the first action location data being within the geofenced area, and causing display of an indicator of the first emergency response entity at the user interface device. The process 500 may also include receiving second user location data associated with the user account, the second user location data indicating that the user account is outside of the geofenced area and receiving second action location data associated with a second emergency response entity. The process 500 may also include generating, based at least in part on the second user location data and second action location data, an updated indicator of the second emergency response entity instead of the indicator of the first emergency response entity at the user interface device.

Additionally, or alternatively, the process 500 may include receiving first location data associated with the user account, receiving second location data associated with an emergency response entity, and selecting the emergency response entity based in part on the first location data and the second location data. The process 500 may also include determining, based at least in part on receiving the user input data, a threshold response time associated with the user input data and causing display of an indicator of the emergency response entity at the user interface device within the threshold response time.

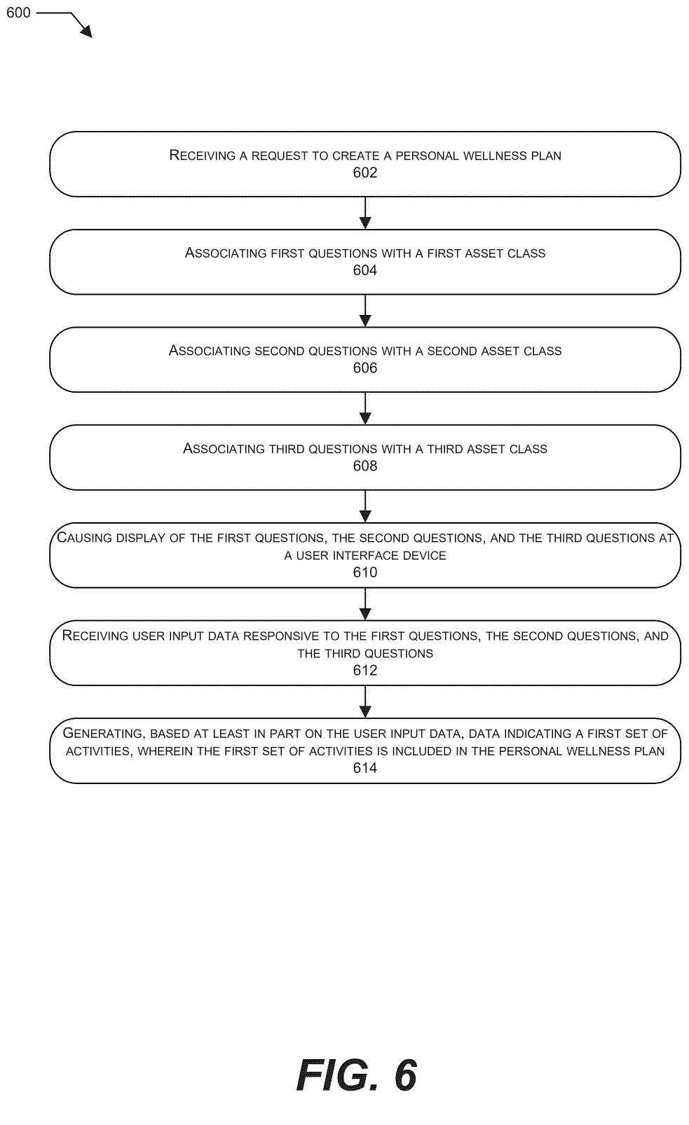

FIG. 6 is a flow diagram of an example process 600 for dynamic activity recommendation output according to an example described herein. The order in which the operations or steps are described is not intended to be construed as a limitation, and any number of the described operations may be combined in any order and/or in parallel to implement process 600.

At block 602, the process 600 may include receiving a request to create a personal wellness plan. For example, a user may desire to create a personal wellness plan and may interact with a user device. For example, the user device may receive user input from the user, indicating a request to create the personal wellness plan as part of making a user account on a mobile application installed on the user device. The user device may include one or more input sensors for receiving user input and/or user activity input. There may be a variety of input sensors capable to detecting user input and/or user activity input, such as an accelerometer, a camara, a microphone, a global position system (e.g., GPS) receiver, etc. In some examples, the user device may communicate with a remote computing device via a network. The network may include any one or a combination of many different types of networks, such as cable networks, the Internet, wireless networks, cellular networks, and other private and/or public networks. In addition, the network may comprise multiple different networks. For example, the user device may utilize a wireless local area network (WLAN) to communicate with a wireless router, which may then route the communication over a public network (e.g., the Internet) to the remote computing device. For example, when the user device receives user input indicating a request to make a personal wellness plan, the user device 106 may communicate user input data to the remote computing device.

At block 604, the process 600 may include associating first questions with a first asset class. In an example, the remote computing device may include at least one memory and one or more processor(s). The memory may include an operating system, application data, and a question determination component for providing recommended questions to the user device. In some examples, the remote computing device may determine questions to be presented to the user via the user device based on receiving the user input data indicating a request to make a personal wellness plan. The question determination component of the remote computing device may store a library of questions. The remote computing device may associate the first questions from the library of questions with the first asset class.

At block 606, the process 600 may include associating second questions with a second asset class. For example, the remote computing device may determine questions to be presented to the user via the user device based on receiving the user input data indicating a request to make a personal wellness plan. The question determination component of the remote computing device may store a library of questions. The remote computing device may associate the second questions from the library of questions with the second asset class.

At block 608, the process 600 may include associating third questions with a third asset class. For example, the remote computing device may determine questions to be presented to the user via the user device based on receiving the user input data indicating a request to make a personal wellness plan. The question determination component of the remote computing device may store a library of questions. The remote computing device may associate the third questions from the library of questions with the third asset class.

At block 610, the process 600 may include causing display of the first questions, the second questions, and the third questions at a user interface device. For example, the question determination component may receive data from the user device, such as user input data, and may determine questions to display, from a library of questions, on the user device to the user. A remote computing device may provide at least one question associated with each asset class to a user device for display on a mobile application.

At block 612, the process 600 may include receiving user input data responsive to the first questions, the second questions, and the third questions. For example, the user may provide user input (e.g., responses) to each of the questions, where the user input is detected by the input sensor(s) of the user device. When the user device receives user input indicating responses to each of the questions, the user device may communicate the user input data representing the responses to the remote computing device.

At block 614, the process 600 may include generating, based at least in part on the user input data, data indicating a first set of activities, wherein the first set of activities is included in the personal wellness plan. For example, the memory associated with the remote computing device may include an activity determination component for providing recommended activities to the user device. The activity determination component may receive data from the user device, such as user input data, and may determine recommended activities to display, from a library of activities, on the user device to the user.

In some examples, the remote computing device may determine activities to be recommended to the user via the user device based on receiving the user input data indicating responses to each of the questions. The activity determination component of the remote computing device may store a library of activities. The remote computing device may associate the activities from the library of activities with the specific asset classes. Additionally, or alternatively, the remote computing device may associate each question in the library of questions with one or more activities from the library of activities.

In an example, based on the user input data representing the responses to each of the questions determined to be presented by the question determination component, the activity determination component may determine the appropriate activities to be presented to the user. The remote computing device may provide activity determination data to the user device for display, indicating at least one activity associated with each asset class.

Additionally, or alternatively, the process 600 may include receiving user activity data responsive to the first set of activities. The process 600 may also include determining, using a machine learning model trained to determine activities to be associated with personal wellness plans, and based at least in part on the user activity data as an input to the machine learning model, a second set of activities, the second set of activities differing at least in part from the first set of activities. The process 600 may also include generating an updated personal wellness plan, the updated personal wellness plan including the second set of activities instead of the first set of activities.

Additionally, or alternatively, the process 600 may include, wherein the user input data is first user input data, associating fourth questions with the first asset class, associating fifth questions with the second asset class, and associating sixth questions with the third asset class. The process 600 may also include causing display of the fourth questions, the fifth questions, and the sixth questions at the user interface device and receiving second user input data responsive to the fourth questions, the fifth questions, and the sixth questions. The process 600 may also include generating, based at least in part on the second user input data, data indicating a second set of activities, wherein the second set of activities is included in the personal wellness plan.

Additionally, or alternatively, the process 600 may include generating a machine learning model configured to determine questions to be displayed at the user interface device, receiving feedback data indicating performance of the machine learning model, generating training data from the feedback data, and training the machine learning model utilizing the training data, wherein a trained machine learning model is generated. The process 600 may also include using the trained machine learning model to determine fourth questions, fifth questions, and sixth questions to be displayed at the user interface device based at least in part on the user input data responsive to the first questions, the second questions, and the third questions.

Additionally, or alternatively, the process 600 may include receiving user location data, receiving activity location data, selecting an activity based at least in part on the user location data and activity location data, and causing display of an indicator of the activity at the user interface device.

Additionally, or alternatively, the process 600 may include determining, based at least in part on receiving the user input data, a threshold amount of action time associated with the user input data and causing display of the indicator of the activity at the user interface device within the threshold amount of action time.

Additionally, or alternatively, the process 600 may include receiving user input data, wherein the user input data includes a request for a desired set of activities, and generating an updated personal wellness plan, the updated personal wellness plan including the desired set of activities.

Additionally, or alternatively, the process 600 may include receiving user activity data that is responsive to the first set of activities, wherein the user activity data is obtained by a sensor on a wearable device.

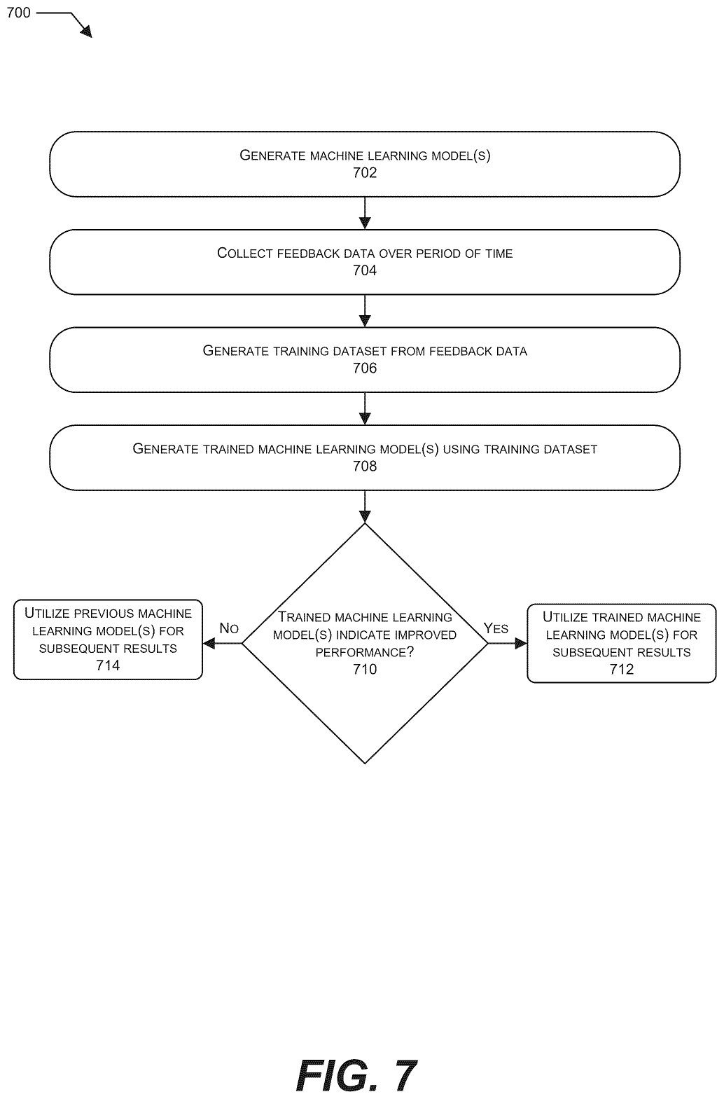

FIG. 7 is a flow diagram of an example process 700 for the generation and training of artificial intelligence models to perform one or more of the processes described herein, according to an example described herein. The order in which the operations or steps are described is not intended to be construed as a limitation, and any number of the described operations may be combined in any order and/or in parallel to implement process 700.

At block 702, the process 700 may include generating one or more artificial intelligence models, such as a machine learning model. A number of artificial intelligence techniques may be employed to generate and/or modify the layers and/or models described herein. Those techniques may include, for example, decision tree learning, association rule learning, artificial neural networks (including, in examples, deep learning), inductive logic programming, support vector machines, clustering, Bayesian networks, reinforcement learning, representation learning, similarity and metric learning, sparse dictionary learning, and/or rules-based artificial intelligence.

At block 704, the process 700 may include collecting feedback data over a period of time. The feedback data may include any data associated with determining questions and/or activities to present to a user, any described with respect to FIGS. 1-6, or any other data that may be utilized to perform the operations described herein. This information may include, for example, user input data, user activity data, etc.

At block 706, the process 700 may include generating a training dataset from the feedback data. Generation of the training dataset may include formatting the feedback data into input vectors for the artificial intelligence model to intake, as well as associating the various data with the outcomes of the questions and/or activities described herein.

At block 708, the process 700 may include generating one or more trained artificial intelligence models utilizing the training dataset. Generation of the trained artificial intelligence models may include updating parameters and/or weightings and/or thresholds utilized by the models to determine appropriate questions to present to the user, appropriate activities to recommend, and the like.

At block 710, the process 700 may include determining whether the trained artificial intelligence models indicate improved performance metrics. For example, a testing group may be generated where the outcomes of given questions and/or activities are known but not to the trained artificial intelligence models. The trained artificial intelligence models may generate results, which may be compared to the known results to determine whether the results of the trained artificial intelligence model produce a superior result than the results of the artificial intelligence model prior to training.

In examples where the trained artificial intelligence models indicate improved performance metrics, the process 700 may include, at block 712, utilizing the trained artificial intelligence models for generating subsequent results. For example, the trained artificial intelligence models may be utilized to determine appropriate questions to present to the user, appropriate activities to recommend, and the like. It should be understood that the trained artificial intelligence models may be utilized in any scenario where models are utilized as described herein.

In examples where the trained artificial intelligence models do not indicate improved performance metrics, the process 700 may include, at block 714, utilizing the previous iteration of the artificial intelligence models for generating subsequent results.

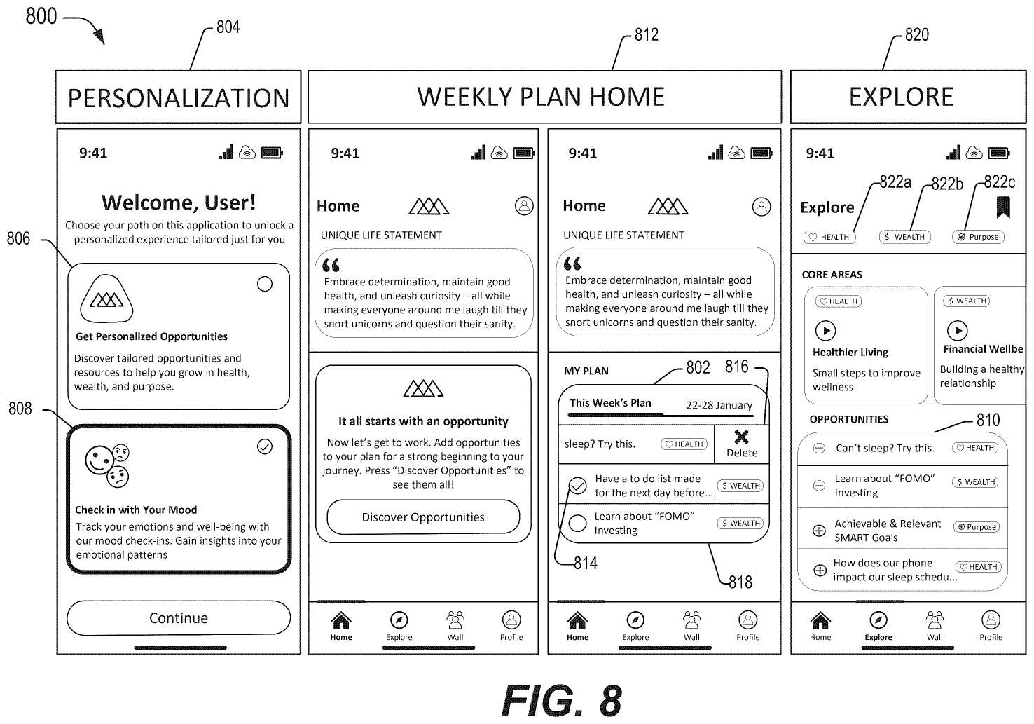

FIG. 8 illustrates an example user interface 800 of the personalized weekly plans 802 in response to user inputs, according to at least some examples of the present disclosure. The example “Personalization” interface 804 may allow the user to select between the Mood Index 806 or mood check-in 808 to facilitate the creation of the personalized weekly plans 802. The Mood Index 806 or mood check-in 808 may inform the creation of an initial set of recommended opportunities 810. The “Weekly Plan Home” interface 812 presents the generated weekly plan 802 and may allow for the management of the weekly plan 802 by allowing the user to add 814 or delete 816 one or more entries 818 from the weekly plan 802. The “Exploration” interface 820 may allow the user to filter opportunities 810 by wellness categories of health 822a, wealth 822b, or purpose 822c. In examples, a user starting with a mood check-in 808 expressing stress may be recommended a “mindfulness meditation” opportunity 810. Over the week, the user may add 814 “yoga for beginners” from the exploration interface 820, setting a goal for three sessions. As the user engages with the selected opportunities 810, the system may learn and suggest a “digital detox challenge” for the following week.

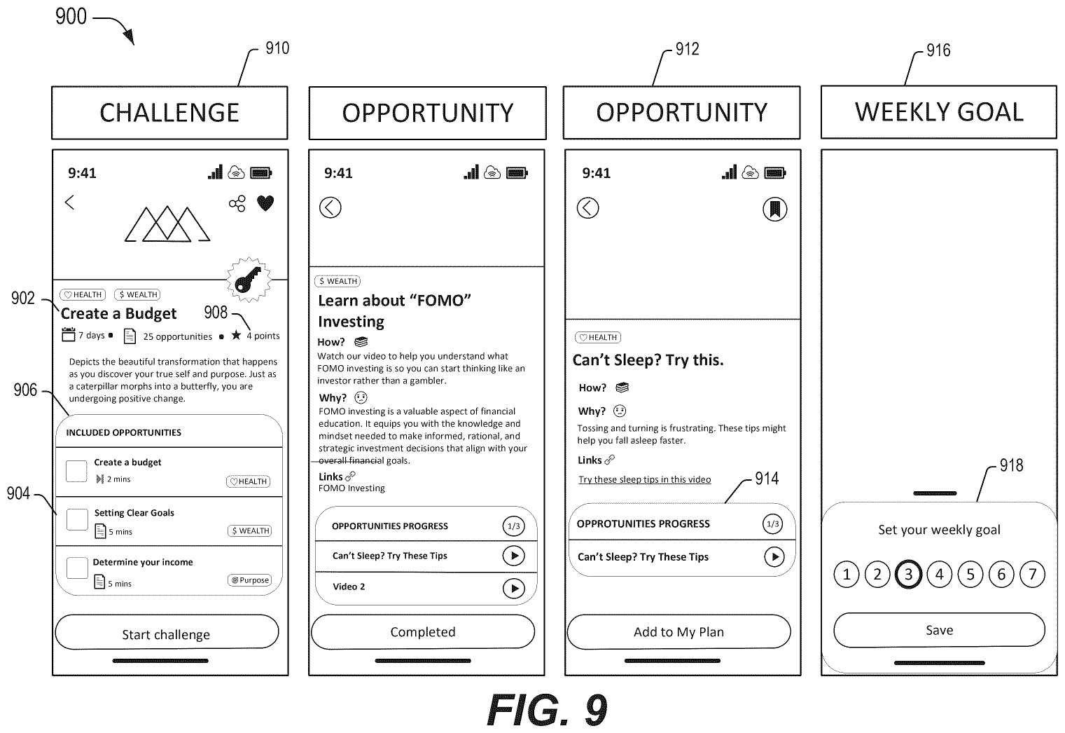

FIG. 9 illustrates an example user interface 900 for the selection of challenges 902 and opportunities 904 integrated into weekly mental wellness plans 906, according to at least some examples of the present disclosure. The “Challenge” interface 910 may allow the user to select generated challenges 902 or opportunities 904 to be integrated into their weekly mental wellness plan 906. The “Opportunities” interface 912 may allow the user to view the opportunities progress 914. The “Weekly Goal” interface 916 may allow the user to set a weekly goal 918. Additionally, upon the completion of challenges 902 or opportunities 904, the user may earn points 908 which may be redeemable for various rewards. In examples, the user may decide to select a “30-day mindfulness” challenge, which includes a daily meditation opportunity, a weekly gratitude opportunity, and a virtual well workshop opportunity. Upon completing each opportunity, the user may earn points which contribute to rewards such as a mindfulness app subscription.

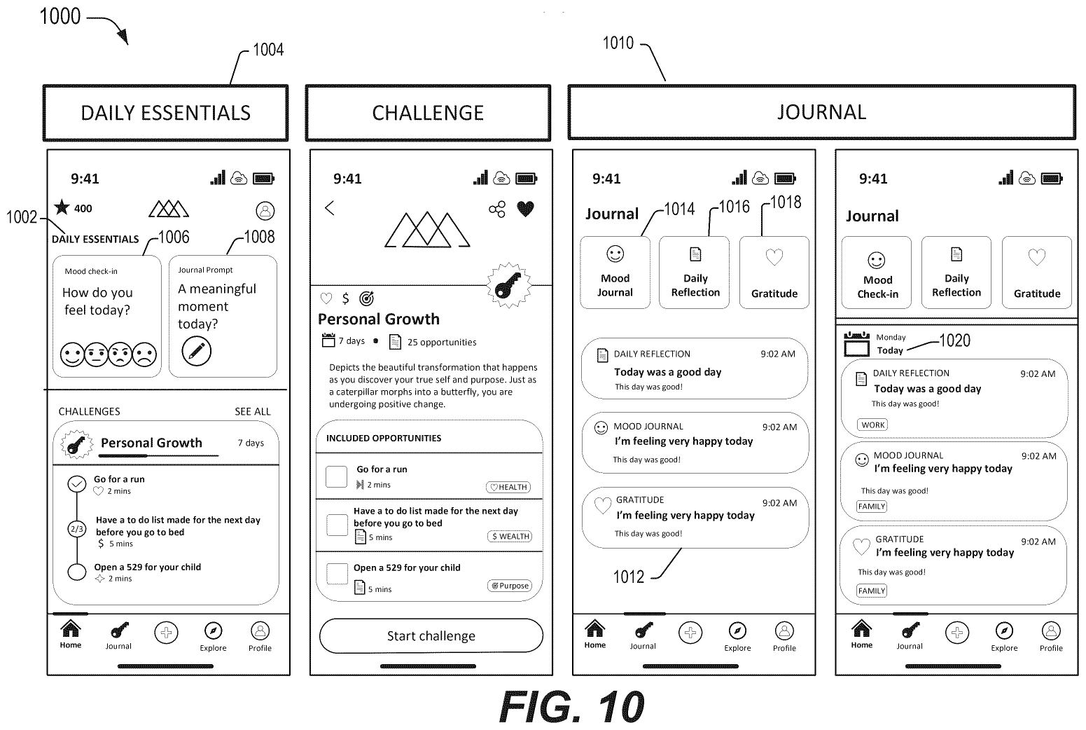

FIG. 10 illustrates an example user interface 1000 of multiple daily essential modules 1002, according to at least some examples of the present disclosure. The “Daily Essentials” interface 1004 may display various daily essential modules 1102, such as a mood check-in 1006 or journaling 1008 module to name a few non limiting examples. The “Journal” interface 1010 may allow the user to create journal entries 1012 tied to specific journal types, such as mood 1014, daily reflection 1016, or gratitude 1018 journal types to name a few nonlimiting examples. Additionally, the “Journal” interface 1010 may compile the created journal entries 1012 into a “today” 1020 list of interactions. The compiled “today” 1020 list of interactions may allow the user to scroll through historical entries for ongoing reflection and mood tracking. In examples, the user may begin their day with the daily essentials, opting to complete a breathwork exercise followed by a gratitude journal entry. The user may reflect on what they are thankful for, tying the journal entry to their morning meditation activity. Over time, the user's use of the journal module may allow for the observation of trends in their entries, identifying factors contributing to positive and negative mood shifts.

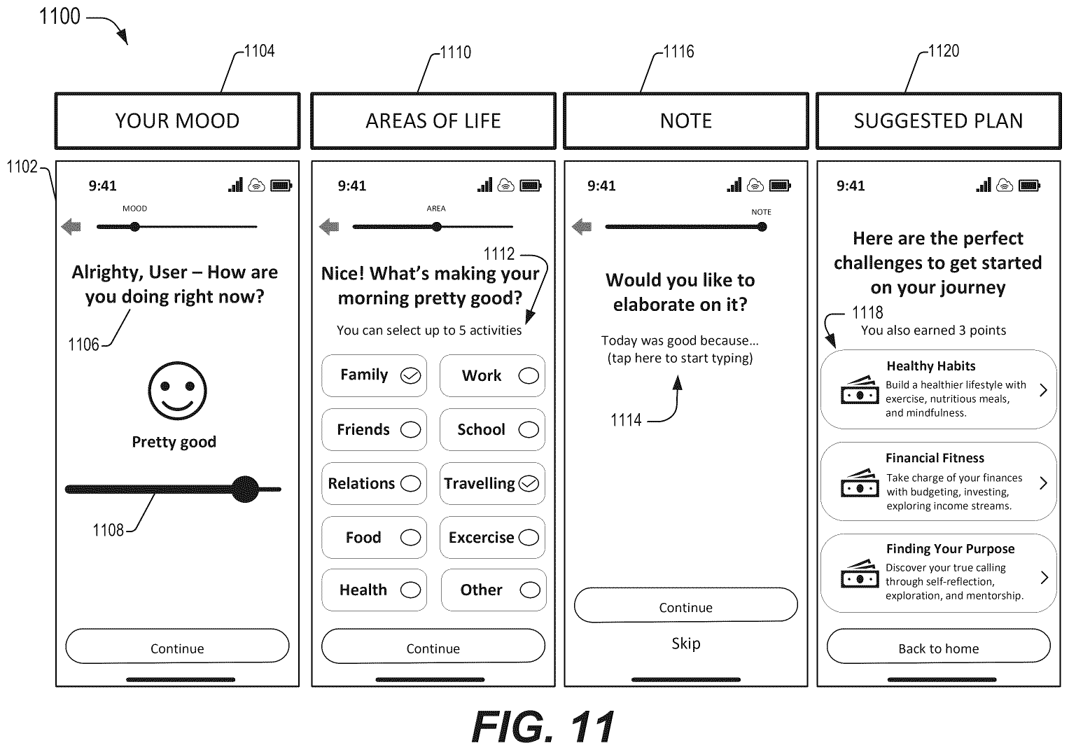

FIG. 11 illustrates an example user interface 1100 of a mood tracker 1102 for collecting user input responses, according to at least some examples of the present disclosure. The “Your Mood” interface 1104 may prompt the user with a question 1106 and provide a slider 1108 for collecting user input responses. The “Areas of Life” interface 1110 may allow the user to select activities 1112 influencing their mood. The user may provide additional details via a manual note pad 1114 displayed on the “Note” interface 1116. Upon collecting user input responses in the mood tracker 1102, a suggested plan 1118 may be provided on the “Suggested Plan” interface 1120. In examples, a user may log a mood of “unhappy,” attributing it to “work” and “heath,” with notes about stress and lack of exercise. The mobile application 114 may then recommend a stress management challenge that has positively impacted users with similar profiles, guiding the user towards activities like mindfulness and physical exercise.

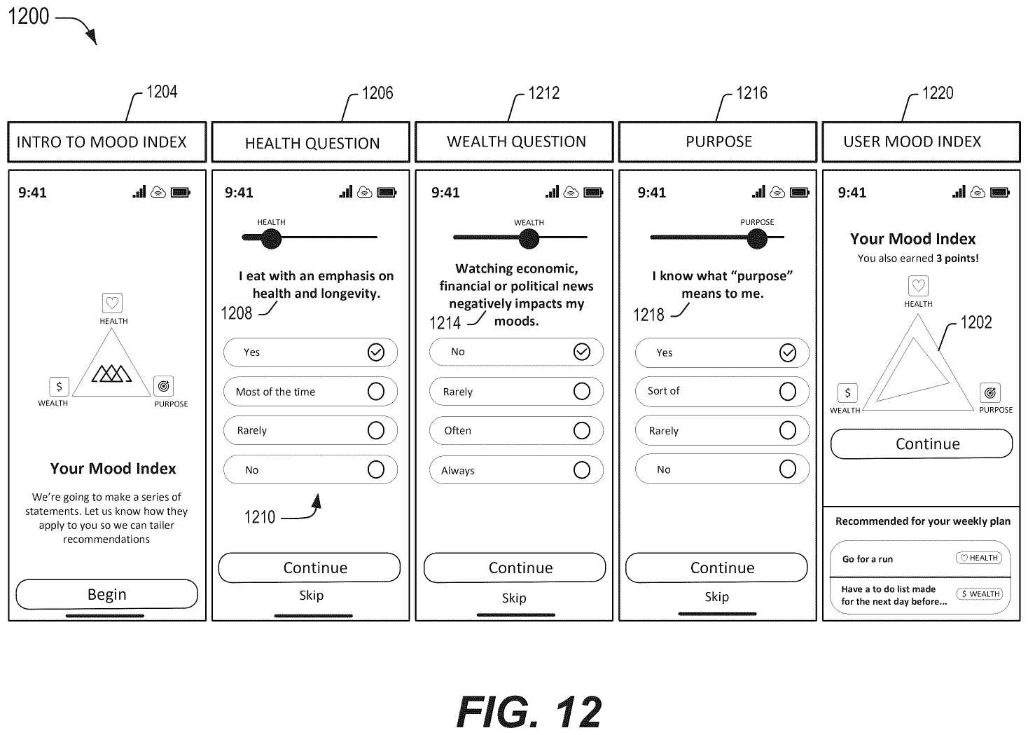

FIG. 12 illustrates an example user interface 1200 of the Mood Index 1202 for tracking mental wellness improvements in response to user inputs, according to at least some examples of the present disclosure. The “Intro To Mood Index” interface 1204 briefs the user on how to complete various questions to complete to Mood Index 1202. The “Health Question” interface 1206 may provide the user with one or more heath related questions 1208. In response to the one or more health related questions 1208, the user may select from the provided answer choices 1210 which best captures their feelings. The “Wealth Question” interface 1212 may provide the user with one or more wealth related questions 1214. In response to the one or more wealth related questions 1214, the user may select from the provided answer choices 1210 which best captures their feelings. The “Purpose” interface 1216 may provide the user with one or more purpose related questions 1218. In response to the one or more purpose related questions 1218, the user may select from the provided answer choices 1210 which best captures their feelings. Upon completion of the one or more health 1208, wealth 1214, and purpose 1218 questions, the user's Mood Index 1202 may be displayed on the “User Mood Index” interface 1220. In examples, the user may retake the Mood Index assessment, revealing significant improvements in the health category. These updates may refine the user's Mood Index 1202 in the health, wealth, and purpose categories. This change in the user's Mood Index 1202 may cause adjustments in the various provided challenges and opportunities.

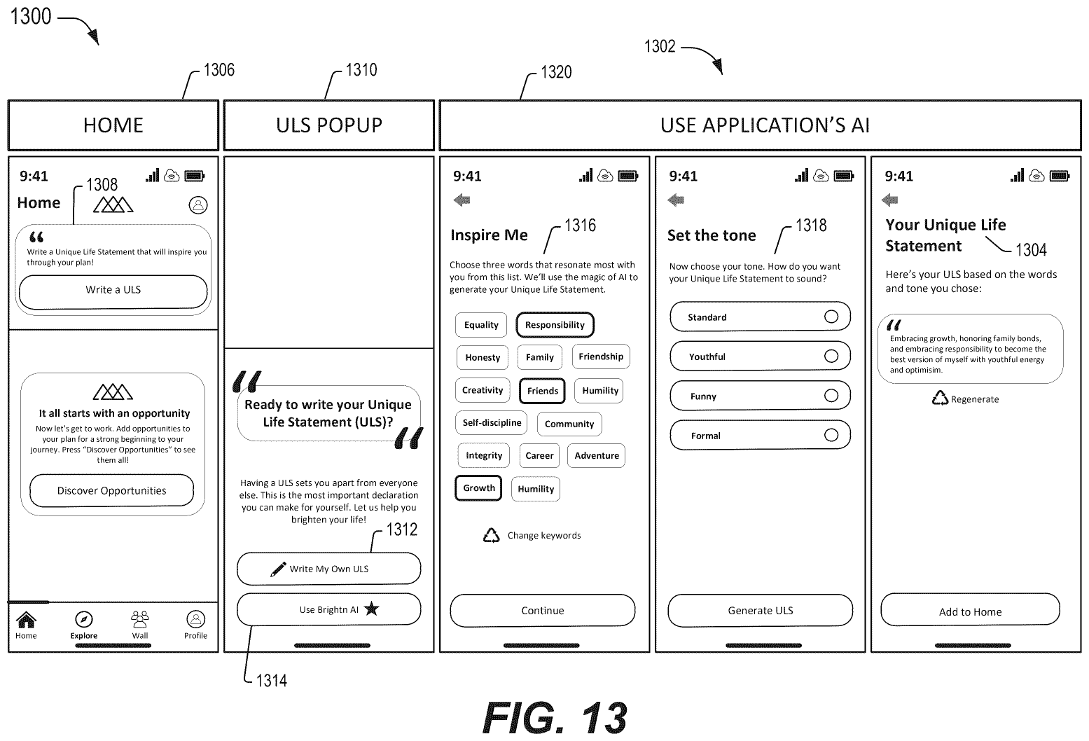

FIG. 13 illustrates an example user interface 1300 of the application AI 1302 for the creation of unique life statements (ULS) 1304 in response to user inputs, according to at least some examples of the present disclosure. The application AI 1302 may allow a user to create a ULS 1304 with the assistance of artificial intelligence. The “Home” interface 1306 may prompt 1308 the user to write a ULS 1304. If the user decides to create a ULS 1304, the “ULS Popup” 1310 may allow the user to write their own ULS 1312 or use the assistance of the application AI 1314. Upon opting to use the assistance of the application AI 1314, the user may be guided through a selection of inspirational key words 1316 and tones 1318 displayed on the “Use Application's AI” interface 1320. The application AI 1302 may then generate a ULS 1304 that is reflective of the user's input and responses. In generating the ULS 1304, the application AI 1302 may analyze user data, including mood, preferences, Mood Index scores, and journal entries, to name a few non-limiting examples. In examples, the user may select family and friendship inspiration key words to receive a ULS that emphasizes the importance of connections and humor. Later, based on the user's engagement, the application AI may recommend a “30-day challenge,” filled with activities to strengthen relationships.

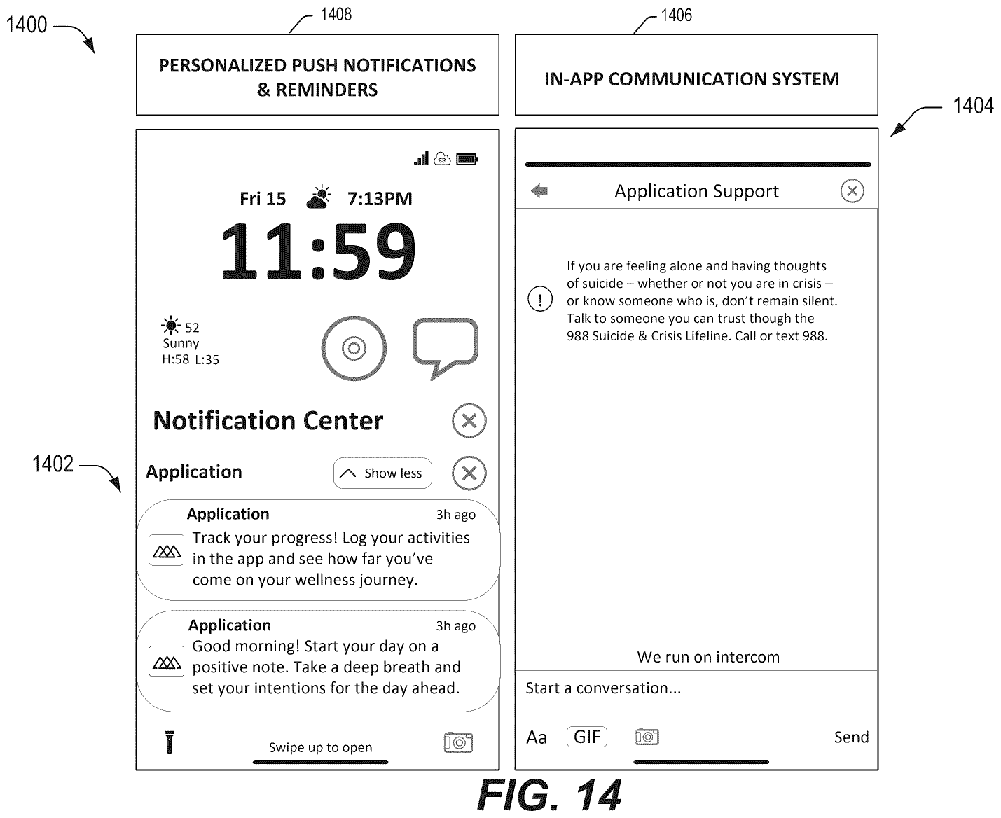

FIG. 14 illustrates an example user interface 1400 of generated push notifications 1402 in response to user inputs in the communication system 1404, according to at least some examples of the present disclosure. The “Personalized Push Notifications & Reminders” interface 1408 may display generated push notifications 1402 in response to user inputs and may prompt user action. The generated push notifications 1402 may be based on user behavior or utilizing various user data (e.g., mood tracker, journal entries, and user inactivity) to name a few non-liming examples. The user may associate their profile with specific organizations or schools using unique codes. The “In-App Communication System” interface 1406 may display and facilitate direct communication with a wellness advisor. In examples, a user who has not logged a journal entry in several days may receive a generated push notification encouraging reflection, with a deep link to the journal module. In other examples, a student entering school may receive customized challenges related to upcoming school wellness events. Additionally, a user struggling with stress, may get an in-app message suggesting a direct chat with a life coach and personalized activities to alleviate stress.

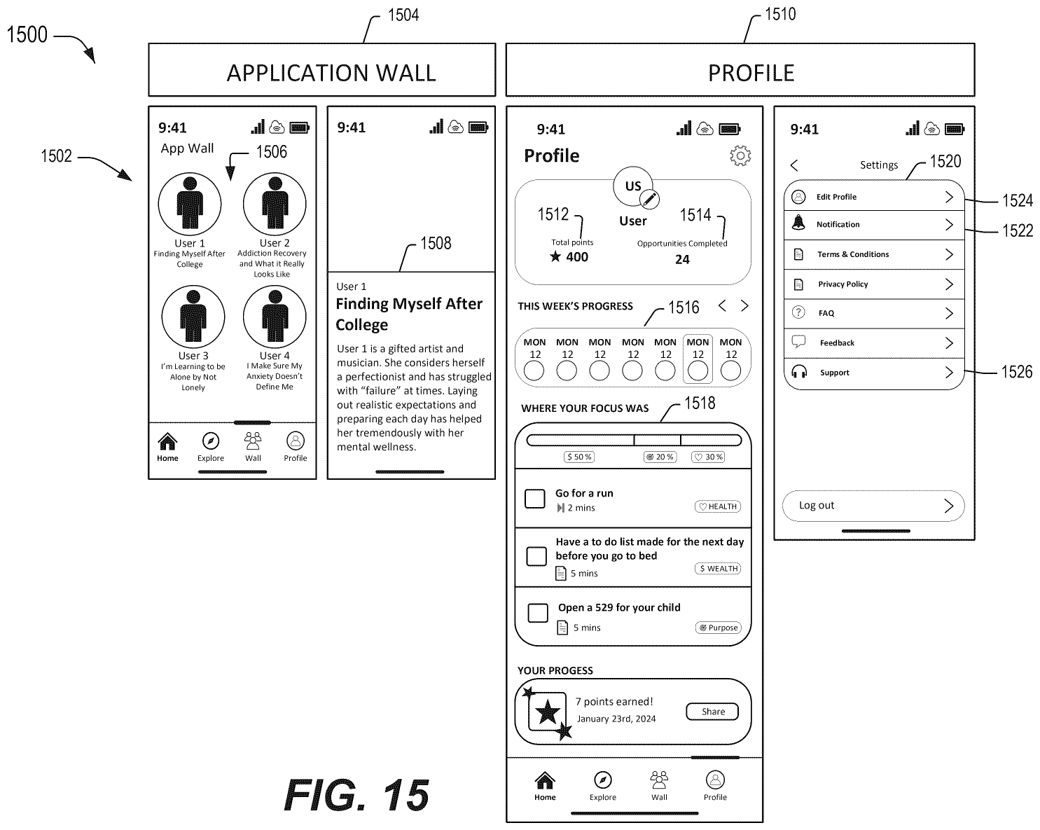

FIG. 15 illustrates an example user interface 1500 of the social platform Application Wall 1502 within the mental wellness application, according to at least some examples of the present disclosure. The “Application Wall” interface 1504 may display one or more user profiles 1506 and user stories 1508. The user may filter user profiles 1506 and user stories 1508 to curate a selection best suited for them. In examples, the “Profile” interface 1510 may display a user's profile information, such as points earned 1512, opportunities completed 1514, weekly progress 1516, and weekly focus 1518, to name a few non-limiting examples. Additionally, “Profile” interface 1510 may display the system settings 1520. The settings 1520, may enable the user to adjust generated notifications 1522, profile settings 1524, and access to support 1526, to name a few non-limiting examples. In examples, the user may share their success story about overcoming anxiety through meditation challenges on the Application Wall 1502. Another user may view their monthly progress in the profile section, noting a significant increase in health activities, and decide to share this achievement with a friend. A third user, feeling overwhelmed, may use the support feature in the settings to schedule a session with a local counselor.

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Although the application describes examples having specific structural features and/or methodological acts, it is to be understood that the claims are not necessarily limited to the specific features or acts described. Rather, the specific features and acts are merely illustrative some examples that fall within the scope of the claims.

What is claimed is:

1. A system, comprising:
one or more processors; and
non-transitory computer-readable media storing computer-executable instructions that, when executed by the one or more processors, cause the one or more processors to perform operations comprising:
receiving a user request to create a personal wellness plan, wherein the personal wellness plan is associated with a user account;
associating a plurality of questions with a plurality of asset classes, the plurality of asset classes including at least a health asset class, a wealth asset class, and a purpose asset class;
selecting a first, second, and third set of questions from the plurality of questions, wherein the first set of questions corresponds to the health asset class, the second set of questions corresponds to the wealth asset class, and the third set of questions corresponds to the purpose asset class;
causing display of the first, second, and third set of questions at a user interface device at a first time;
receiving user input data representing a response to the first, second, and third set of questions;
generating, based at least in part on the user input data, a first set of activities from a plurality of activities, wherein the first set of activities is included in the personal wellness plan;
receiving user activity data that is responsive to the first set of activities;
determining, using a machine learning model and based at least in part on the user activity data, a second set of activities from the plurality of activities; and
updating the personal wellness plan associated with the user account to include the second set of activities instead of the first set of activities.

2. The system of claim 1, wherein the user input data is first user input data and the user activity data is first user activity data, the operations further comprising:
selecting, using the machine learning model and based at least in part on the first user input data, a fourth, fifth, and sixth set of questions from the plurality of questions, wherein the fourth set of questions corresponds to the health asset class, the fifth set of questions corresponds to the wealth asset class, and the sixth set of questions corresponds to the purpose asset class;
causing display of the fourth, fifth, and sixth set of questions at the user interface device at a second time;
receiving second user input data representing a response to the fourth, fifth, and sixth set of questions;
generating, based at least in part on the second user input data, a third set of activities from the plurality of activities, wherein the third set of activities is included in the personal wellness plan;
receiving second user activity data that is responsive to the third set of activities;
determining, using the machine learning model and based at least in part on the second user activity data, a fourth set of activities from the plurality of activities; and
updating the personal wellness plan associated with the user account to include the fourth set of activities instead of the third set of activities.

3. The system of claim 1, the operations further comprising:
receiving first location data associated with the user account;
receiving second location data associated with an emergency response;
determining, based in part on the first location data and the second location data, that the user account is proximate to the emergency response; and
causing display of the emergency response at the user interface device.

4. The system of claim 3, the operations further comprising:
determining, in response to receiving user input data, a threshold amount of required response time associated with the user input data; and
causing display of the emergency response at the user interface device within the threshold amount of required response time.

5. A method, comprising:
receiving a request to create a personal wellness plan;
associating a plurality of questions with a plurality of asset classes, the plurality of asset classes including at least a first, second, and third asset class;
selecting a set of questions from the plurality of questions, wherein the set of questions corresponds to each one of the first, second, and third asset class;
causing display of the set of questions at a user interface device;
receiving user input data representing a response to the set of questions; and
generating, based at least in part on the user input data, a first set of activities from a plurality of activities, wherein the first set of activities is included in the personal wellness plan.

6. The method of claim 5, further comprising:
receiving user activity data that is responsive to the first set of activities;
determining, using a machine learning model and based at least in part on the user activity data, a second set of activities from the plurality of activities; and
updating the personal wellness plan to include the second set of activities instead of the first set of activities.

7. The method of claim 5, wherein the set of questions is a first set of questions and the user input data is first user input data, further comprising:
determining, using a machine learning model based at least in part on the user input data, a second set of questions from the plurality of questions;
causing display of the second set of questions at the user interface device;
receiving second user input data representing a response to the second set of questions; and
generating, based at least in part on the second user input data, a second set of activities from the plurality of activities, wherein the second set of activities is included in the personal wellness plan.

8. The method of claim 5, wherein the set of questions is a first, second, and third set of questions, further comprising:
associating the plurality of questions with the plurality of asset classes, wherein the plurality of asset classes includes at least a health asset class, a wealth asset class, and a purpose asset class; and
selecting the first, second, and third set of questions from the plurality of questions, wherein the first set of questions corresponds to the health asset class, the second set of questions corresponds to the wealth asset class, and the third set of questions corresponds to the purpose asset class.

9. The method of claim 5, further comprising:
receiving user location data;
receiving activity location data;
determining, based in part on the user location data and activity location data, that a user is in proximity to an activity; and
causing display of the activity at the user interface device.

10. The method of claim 9, further comprising:
determining, in response to receiving user input data, a threshold amount of action time associated with the user input data; and
causing display of the activity at the user interface device within the threshold amount of action time.

11. The method of claim 5, further comprising:
receiving user input data, wherein the user input data includes a request for a desired set of activities; and
updating the personal wellness plan to include the desired set of activities.

12. The method of claim 5, further comprising:
receiving user activity data that is responsive to the first set of activities, wherein the user activity data is obtained by a sensor on a wearable device.

13. A system, comprising:
one or more processors; and
non-transitory computer-readable media storing computer-executable instructions that, when executed by the one or more processors, cause the one or more processors to perform operations comprising:
receiving a request to create a personal wellness plan;
associating a plurality of questions with a plurality of asset classes, the plurality of asset classes including at least a first, second, and third asset class;
selecting a set of questions from the plurality of questions, wherein the set of questions corresponds to each one of the first, second, and third asset class;
causing display of the set of questions at a user interface device;
receiving user input data representing a response to the set of questions; and
generating, based at least in part on the user input data, a first set of activities from a plurality of activities, wherein the first set of activities is included in the personal wellness plan.

14. The system of claim 13, the operations further comprising:
receiving user activity data that is responsive to the first set of activities;
determining, using a machine learning model and based at least in part on the user activity data, a second set of activities from the plurality of activities; and
updating the personal wellness plan to include the second set of activities instead of the first set of activities.

15. The system of claim 13, wherein the set of questions is a first set of questions and the user input data is first user input data, the operations further comprising:
determining, using a machine learning model based at least in part on the user input data, a second set of questions from the plurality of questions;
causing display of the second set of questions at the user interface device;
receiving second user input data representing a response to the second set of questions; and
generating, based at least in part on the second user input data, a second set of activities from the plurality of activities, wherein the second set of activities is included in the personal wellness plan.

16. The system of claim 13, wherein the set of questions is a first, second, and third set of questions, the operations further comprising:
associating the plurality of questions with the plurality of asset classes, wherein the plurality of asset classes includes at least a health asset class, a wealth asset class, and a purpose asset class; and
selecting the first, second, and third set of questions from the plurality of questions, wherein the first set of questions corresponds to the health asset class, the second set of questions corresponds to the wealth asset class, and the third set of questions corresponds to the purpose asset class.

17. The system of claim 13, the operations further comprising:
receiving user location data;
receiving activity location data;
determining, based in part on the user location data and activity location data, that a user is in proximity to an activity; and
causing display of the activity at the user interface device.

18. The system of claim 17, the operations further comprising:
determining, in response to receiving user input data, a threshold amount of action time associated with the user input data; and
causing display of the activity at the user interface device within the threshold amount of action time.

19. The system of claim 13, the operations further comprising:
receiving user input data, wherein the user input data includes a request for a desired set of activities; and
updating the personal wellness plan to include the desired set of activities.

20. The system of claim 13, the operations further comprising:
receiving user activity data that is responsive to the first set of activities, wherein the user activity data is obtained by a sensor on a wearable device.
